(19) United States
(12) Patent Application Publication
(10) Pub. No.: US 2026/0298943 A1
(43) Pub. Date: Oct. 1, 2026

(54) METHODS OF DETERMINING RELATIVE UNPAIRED GLYCAN CONTENT

(71) Applicant: AMGEN INC., Thousand Oaks, CA (US)

(72) Inventors: Esme CANDISH, Somerville, MA (US); Alla POLOZOVA, North Kingstown, RI (US)

(73) Assignee: AMGEN INC., Thousand Oaks, CA (US)

(21) Appl. No.: 19/475,810

(22) PCT Filed: Apr. 19, 2024
(86) PCT No.: PCT/US2024/025561

§ 371 Date: Oct. 16, 2025

Related U.S. Application Data
(60) Provisional application No. 63/576,448, filed on Apr. 20, 2023.

(51) Int. Cl. G01N 33/68; C07K 16/28

(52) U.S. Cl. CPC G01N 33/6857; C07K 16/283; G01N 33/6848; C07K 2317/41; C07K 2317/53; C07K 2317/55; C07K 2317/732; G01N 2333/942; G01N 2333/952

(57) ABSTRACT

Methods of determining relative unpaired glycan content of an IgG antibody composition are provided. In exemplary embodiments, the method comprises treating the IgG antibody composition with two enzymes to forma mixture of Fab fragments and Fc fragments, wherein one enzyme cleaves an antibody heavy chain at a site N-terminal to the hinge region disulfide linkages and the other enzyme cleaves the β1,4 linkage between core GlcNAc residues to form Fc fragments each comprising a pair of core glycan structures, (b) separating the Fab fragments from the Fc fragments, and (c) quantifying the abundance of (i) paired afucosylated Fc fragments, (ii) unpaired afucosylated Fc fragments, (iii) paired high mannose Fc fragments, and/or (iv) unpaired high mannose Fc fragments, to determine the relative unpaired afucosylated (AF) glycan content and relative unpaired high mannose (HM) glycan content.

### CROSS-REFERENCE TO RELATED APPLICATION

The benefit under 35 U.S.C. § 119(e) of U.S. Provisional Patent Application No. 63/576,448, filed on Apr. 20, 2023, is hereby claimed, and the disclosure thereof is hereby incorporated by reference herein.

### BACKGROUND

Glycosylation plays a role in multiple cellular functions, including, for example, protein folding, quality control, molecular trafficking and sorting, and cell surface receptor interaction. The glycan structure of a protein drug affects its therapeutic efficacy, impacting its bioactivity, pharmacokinetics, immunogenicity, solubility, and in vivo clearance. Fc glycoform profiles are product quality attributes for recombinant antibodies, since they directly impact the clinical efficacy and pharmacokinetics of the antibodies. See, e.g., Reusch and Tejada, Glycobiology 25(12): 1325-1334 (2015); and Boune et al., Antibodies (Basel) 9(2): 22 (2020).

Given their importance, the glycan structures attached to recombinant protein drugs are often monitored during development and manufacturing (Li et al., Front Immunol 8: 1554 (2017). Traditionally, glycans are monitored using a released glycan assay, in which glycans are cleaved from the protein backbone, chromatographically separated, and identified. Techniques used for the analysis of immunoglobulin glycosylation are reviewed in de Haan et al., Glycobiology 30(4): 226-240 (2020). The glycan population of IgG therapeutics is innately heterogenous and complex, as glycosylation requires the coordinated functions of multiple metabolic enzymes and is complicated by the linkage of sugar isomers, the glycosylation site occupancy, as well as the outer arm sugar addition, which can include one or more of fucose, galactose, bisecting GlcNAc, and sialic acid. Further diversification of the antibody glycan population is due to the asymmetry of the two N-glycans from every heavy chain and the involvement of random pairing of two different heavy chain glycans.

Even when successful, the current methods used for glycan analysis ignore the fact that many therapeutic protein molecules, including IgG and fusion proteins with IgG Fc region, contain two glycosylated Fc chains. Because the current commonly used methods include cleavage of the glycans from the protein backbone, such methods fail to impart knowledge on the pairing status of the glycan species within individual protein molecules. For example, while current methods may be used to determine the overall abundance of afucosylated glycans in a given antibody composition sample, it does not inform the level of antibodies comprising a fully afucosylated pair of heavy chains vs. comprising only a hemi-afucosylated pair of heavy chains. Such information on pairing status of the protein is suggested to be an important feature of an antibody composition that influences its biological activity level (see, e.g., International Patent Application Publication No. WO 2022/081824).

Glycan pairing analysis is challenging due to the size of the molecules and the highly heterogeneous glycan population. The analysis of specifically afucosylated glycan pairs is further complicated by the low abundance of afucosylated and high mannose glycans relative to other glycans. Accordingly, determining the abundance of afucosylated glycan pairs and teasing out the relative abundance of fully afucosylated pairs or hemi-afucosylated pairs is complex and time-consuming.

Thus, there is a need in the biopharmaceutical industry for simple, efficient and reliable methods of determining unpaired glycan content of an IgG antibody composition.

### SUMMARY

Presented herein are data demonstrating the feasibility and advantages of an improved method of determining the relative unpaired glycan content of an IgG antibody composition. The method includes preparation of a simplified sample and the analysis thereof, which, ultimately, provides for an easier, faster, and more accurate way of determining the relative unpaired glycan content of an IgG antibody composition.

Accordingly, the present disclosure provides methods of determining the relative unpaired glycan content of an IgG antibody composition. In exemplary embodiments, the method comprises (a) treating the IgG antibody composition with two enzymes to form a mixture of Fab fragments and Fc fragments, wherein one enzyme cleaves an antibody heavy chain at a site N-terminal to the hinge region disulfide linkages and the other enzyme cleaves the β1,4 linkage between core GlcNAc residues to form Fc fragments each comprising a pair of core glycan structures, (b) separating the Fab fragments from the Fc fragments, and (c) quantifying the abundance of (i) paired afucosylated Fc fragments, (ii) unpaired afucosylated Fc fragments, (iii) paired high mannose Fc fragments, and/or (iv) unpaired high mannose Fc fragments, to determine the relative unpaired afucosylated (AF) glycan content and relative unpaired high mannose (HM) glycan content of the IgG antibody composition.

The present disclosure also provides methods of preparing an IgG antibody composition having an ADCC activity within a target range, e.g., predetermined target range. In exemplary embodiments, the method comprises determining the relative unpaired glycan content of a sample of the IgG antibody composition in accordance with any one of the presently disclosed methods of determining the relative unpaired glycan content. The relative unpaired AF glycan content and/or relative unpaired HM glycan content of the IgG antibody composition may be compared to a target range of unpaired AF glycan content and/or relative unpaired HM glycan content. For example, the target range may be a reference level of a specification, or of a reference IgG antibody composition. Also, for instance, the target range of unpaired AF glycan content and/or relative unpaired HM glycan content may be a target range of unpaired AF glycan content and/or relative unpaired HM glycan content which statistically correlates with a target range of ADCC activity levels of a reference IgG antibody composition.

Further provided are methods of analyzing an IgG antibody composition. In exemplary embodiments, the method comprises determining the relative unpaired glycan content of a sample of the IgG antibody composition in accordance with any one of the presently disclosed methods of determining the relative unpaired glycan content. In various aspects, the method comprises comparing the relative unpaired AF glycan content and/or relative unpaired HM glycan content of the IgG antibody composition to the relative unpaired AF glycan content and/or relative unpaired HM glycan content of a reference product. The method may be performed in manufacturing, for example to determine if a lot of the IgG antibody composition meets a specification.

Additionally provided are methods of monitoring production of an IgG antibody composition. In exemplary embodiments, the method comprises determining the relative unpaired glycan content of an IgG antibody composition in accordance with any one of the presently disclosed methods of determining the relative unpaired glycan content, for a first sample obtained at a first timepoint and for a second sample taken at a second timepoint which is different from the first timepoint. In exemplary aspects, the method comprises comparing the relative unpaired AF glycan content and/or relative unpaired HM glycan content of the first sample to the relative unpaired AF glycan content and/or relative unpaired HM glycan content of the second sample.

Methods of producing an antibody composition are also provided by the present disclosure. In exemplary embodiments, the method comprises determining relative unpaired glycan content of the antibody composition is determined in accordance with any one of the presently disclosed methods of determining the relative unpaired glycan content, wherein, when the relative unpaired glycan content is determined as outside a predetermined target range, the method further comprises modifying one or more conditions of the cell culture to obtain a modified cell culture and determining the relative unpaired glycan content. The method in various instances comprises repeating steps of the method until the relative unpaired glycan content is within the predetermined target range.

Additional exemplary embodiments and aspects of the present disclosure are provided below.

### BRIEF DESCRIPTION OF THE DRAWINGS

FIG. 1A is an illustration of exemplary glycan structures.

FIG. 1B is an illustration of exemplary glycan groups.

FIG. 2 is a diagram of the salvage pathway and the de novo pathway of fucose metabolism.

FIG. 3A is a representative glycan map chromatogram (full scale view; y-axis max ^{~}440.00 EU)) obtained through HILIC. FIG. 3B is a representative glycan map chromatogram (expanded scale view; y-axis max ^{~}44.00 EU) obtained through HILIC.

FIG. 4A is a series of drawings exemplifying antibodies with unpaired or paired afucosylated glycans and unpaired or paired high mannose glycans. FIG. 4B provides exemplary flow charts useful for assigning nomenclature to a glycan pair comprising canonical glycans. As shown in the exemplary flow chart of Box 1, if the glycan pair comprises zero afucosylated glycans (e.g., both glycans are fucosylated), then the glycan pair is “paired fucosylated”. If both glycans of the glycan pair are afucosylated, then the glycan pair is “paired afucosylated”. If only one glycan is afucosylated, then the glycan pair is “unpaired afucosylated”. As shown in the exemplary flow chart of Box 2, if the glycan pair comprises zero high mannose glycans, then Box 1 is used for determining glycan pair classification. If both glycans comprise a high mannose and are afucosylated, then the glycan pair is “paired high mannose”. If only one glycan of the glycan pair comprises a high mannose and is afucosylated, and the other glycan lacks a high mannose and is fucosylated, then the glycan pair is “unpaired high mannose”. If only one glycan of the glycan pair comprises a high mannose and the other chain is afucosylated, then the glycan pair is “paired afucosylated”. From the perspective of high mannose, this glycan pair is “unpaired high mannose”. Additional information useful for assigning nomenclature to a glycan pair is provided herein. See, e.g., Example 1.

FIG. 5 is an illustration of a two-enzyme digestion.

FIG. 6A shows exemplary chromatographic peaks obtained from a 95 mi HILIC-MS separation, wherein Fab fragments are separated from glycosylated Fc fragments. The inset shows a zoomed view of the glycosylated Fc fragment peaks. FIG. 6B shows a further zoomed view of the glycosylated Fc fragment peaks. The glycopair identity for each peak is noted. Those labeled in bold indicate a relevant glycopair.

FIG. 7 shows examples of an unpaired afucoslyated Fc fragment, paired afucosylated Fc fragment, unpaired high mannose Fc fragment, and paired high mannose Fc fragment, following a two-enzyme digestion.

FIG. 8A is an illustration of the simplified chromatographic separation of Fab fragments from Fc fragments, the extraction of the one Fc fragment chromatographic peak for mass spectroscopy, and mass spectra of the Fc fragment chromatographic peak. FIG. 8B shows deconvoluted mass spectra (left) and a zoomed view thereof (right). Deconvoluted mass spectra peaks are matched to a database for peak identification.

Each of FIGS. 9A-9C is a deconvoluted spectrum of samples of the panel described in Example 2. Peaks for relevant glycoform species are shown.

FIG. 10 is a schematic of a cell-based ADCC assay.

FIG. 11A is leverage plot of relative ADCC activity level (%) as measured by the cell-based ADCC assay plotted as a function of measured released afucosylated glycans (%). The best fit line is the solid diagonal line in the middle of the shaded area. p<0.0001.

FIG. 11B is leverage plot of relative ADCC activity level (%) as measured by the cell-based ADCC assay plotted as a function of measured released high mannose glycans (%). The best fit line is the solid diagonal line in the middle of the shaded area. p=0.2786.

FIG. 11C is graph of the Actual ADCC activity level (%6) as measured by the cell-based ADCC assay plotted against the Predicted ADCC activity level (%) as calculated using Equation 8.

FIG. 12A is leverage plot of relative ADCC activity level (%) as measured by the cell-based ADCC assay plotted as a function of measured unpaired afucosylated glycans (%). The best fit line is the solid diagonal line in the middle of the shaded area. p<0.0001.

FIG. 12B is leverage plot of relative ADCC activity level (%) as measured by the cell-based ADCC assay plotted as a function of measured unpaired high mannose glycans (%6). The best fit line is the solid diagonal line in the middle of the shaded area. p=0.0012.

FIG. 12C is graph of the Actual ADCC activity level (%) as measured by the cell-based ADCC assay plotted against the Predicted ADCC activity level (%) as calculated using Equation 5.

### DETAILED DESCRIPTION

The present disclosure provides methods of determining the relative unpaired glycan content of an IgG antibody composition. In exemplary embodiments, the method comprises treating the IgG antibody composition with two enzymes to form a mixture of Fab fragments and Fc fragments, wherein one enzyme cleaves an antibody heavy chain at a site N-terminal to the hinge region disulfide linkages and the other enzyme cleaves the β1,4 linkage between core GlcNAc residues to form Fc fragments each comprising a pair of core glycan structures, (b) separating the Fab fragments from the Fc fragments, and (c) quantifying the abundance of (i) paired afucosylated Fc fragments, (ii) unpaired afucosylated Fc fragments, (iii) paired high mannose Fc fragments, and/or (iv) unpaired high mannose Fc fragments, to determine the relative unpaired afucosylated (AF) glycan content and relative unpaired high mannose (HM) glycan content. As discussed further below, the present inventive methods may be used for preparing an IgG composition having an ADCC activity within a target range, analyzing an IgG antibody composition, monitoring production of an IgG antibody composition, and/or producing an antibody composition.

### Glycosylation, Glycans, and Methods of Glycon Measurement

Many secreted proteins undergo post-translational glycosylation, a process by which sugar moieties (e.g., glycans, saccharides) are covalently attached to specific amino acids of a protein. In eukaryotic cells, two types of glycosylation reactions occur: (1) N-linked glycosylation, in which glycans are attached to the asparagine of the recognition sequence Asn-X-Thr/Ser, where “X” is any amino acid except proline, and (2) O-linked glycosylation in which glycans are attached to serine or threonine. Regardless of the glycosylation type (N-linked or O-linked), microheterogeneity of protein glycoforms exists due to the large range of glycan structures associated with each site (0 or N).

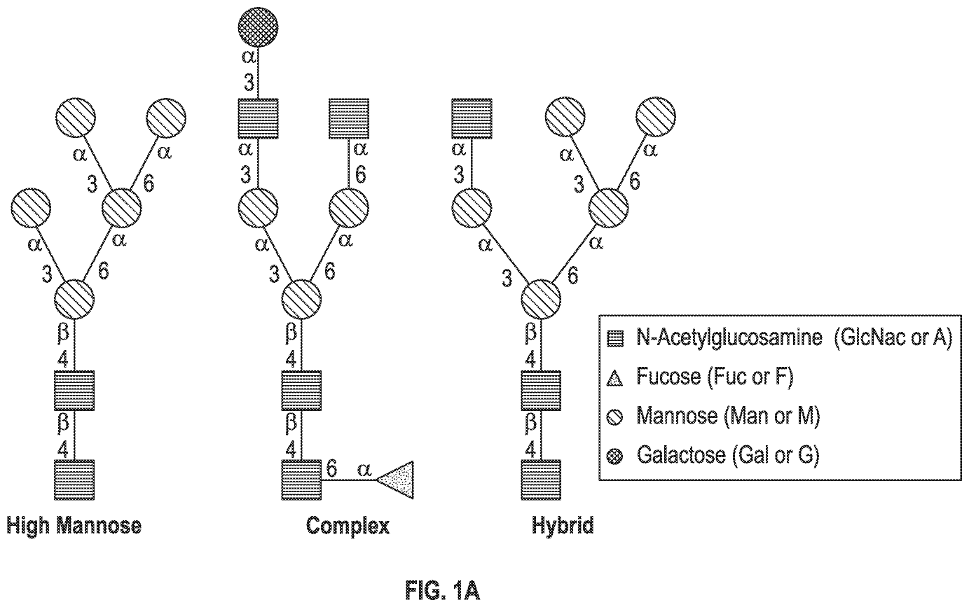

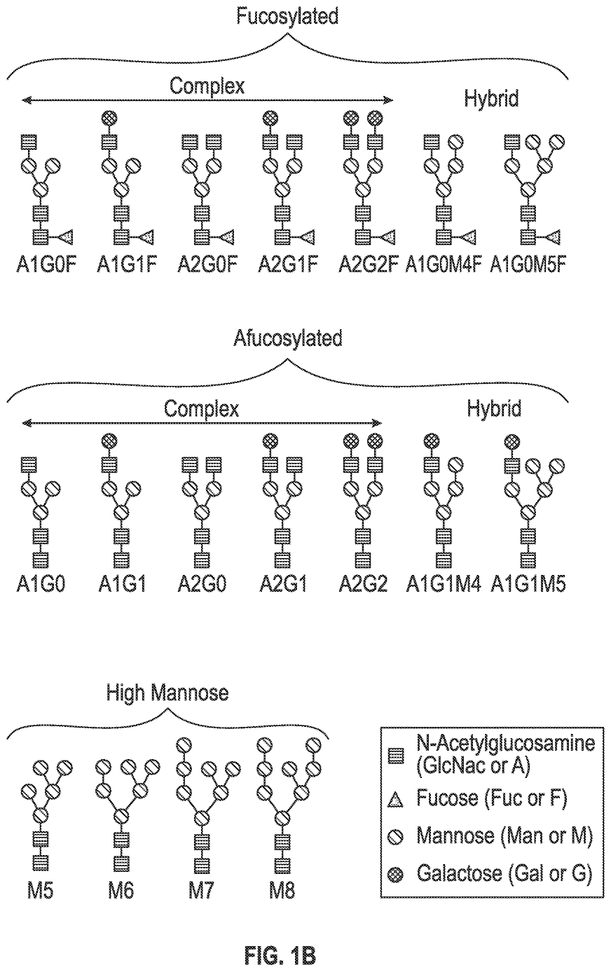

All N-glycans have a common core sugar sequence: Manα1-6(Manα1-3)Manβ1-4GlcNAcβ1-4GlcNAcβ1-Asn-X-Ser/Thr (Man₃GlcNAc₂Asn) and are categorized into one of three types: (A) a high mannose (HM) or oligomannose (OM) type, which consists of two N-acetylglucosamine (GalNAc) moieties and at least 5 (e.g., 5, 6, 7, 8 or 9) mannose (Man) residues, (B) a complex type, which comprises more than two GlcNAc moieties and any number of other sugar types, or (C) a hybrid type, which comprises a Man residue on one side of the branch and GlcNAc at the base of a complex branch. FIG. 1A (adapted from Stanley et al., Chapter 8: N-Glycans, Essentials of Glycobiology, 2′ ed., Cold Spring Harbor Laboratory Press; 2009) shows the three types of N-glycans.

N-linked glycans found in IgG molecules typically comprise one or more monosaccharides of galactose (Gal), N-glucose (Glc), N-acetylglucoasamine (GlcNAc), glucoasamine (GlcN), mannose (Man), fucose (Fuc). Exemplary glycans, their identity and group classifications are shown in FIG. 1B.

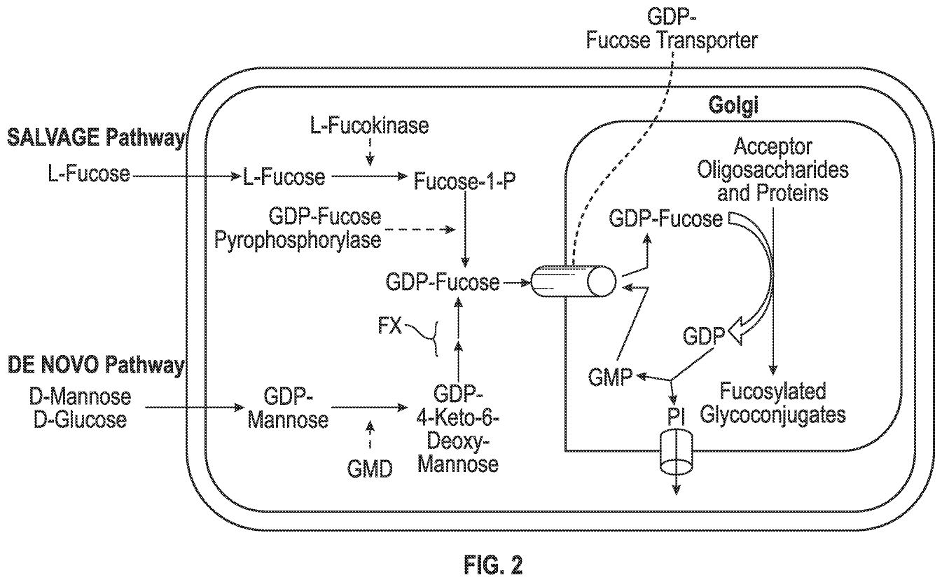

N-linked glycosylation begins in the endoplasmic reticulum (ER), where a complex set of reactions result in the attachment of a core glycan structure made essentially of two GlcNAc residues and three Man residues. The glycan complex formed in the ER is modified by action of enzymes in the Golgi apparatus. If the saccharide is relatively inaccessible to the enzymes, it typically stays in the original HM form. If enzymes can access the saccharide, then many of the Man residues are cleaved off and the saccharide is further modified, resulting in the complex type N-glycans structure. For example, mannosidase-1 located in the cis-Golgi, can cleave or hydrolyze a HM glycan, while fucosyltransferase FUT-8, located in the medial-Golgi, fucosylates the glycan (Hanrue Imai-Nishiya (2007), BMC Biotechnology, 7:84). Fucose metabolism is accomplished through the salvage pathway or the de novo pathway as shown in the diagram of FIG. 2. In the salvage pathway, free L-fucose is converted to GDP-fucose, while in the de novo pathway, GDP-fucose is synthesized via three reactions catalyzed by GMD and FX. GDP-fucose is then transported from the cytosol to the Golgi lumen by GDP-Fuc Transferase and transferred to acceptor oligosaccharides and proteins. The other reaction product, GDP, is converted by a luminal nucleotide diphosphatase to guanosine 5-monophosphate (GMP) and inorganic phosphate (Pi). The former is exported to the cytosol (via an antiport system that is coupled with the transport of GDP-fucose), whereas the latter is postulated to leave the Golgi lumen via the Golgi anion channel, GOLAC. See, e.g., Nordeen et al. 2000; Hirschberg et al. 2001.

Accordingly, the sugar composition and the structural configuration of a glycan structure varies, depending on the glycosylation machinery in the ER and the Golgi apparatus, the accessibility of the machinery enzymes to the glycan structure, the order of action of each enzyme and the stage at which the protein is released from the glycosylation machinery, among other factors.

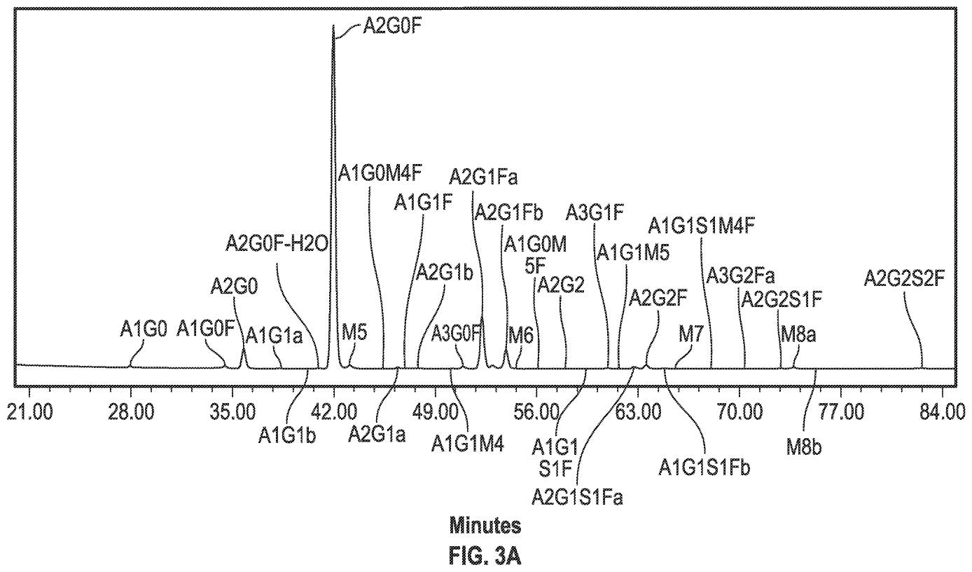

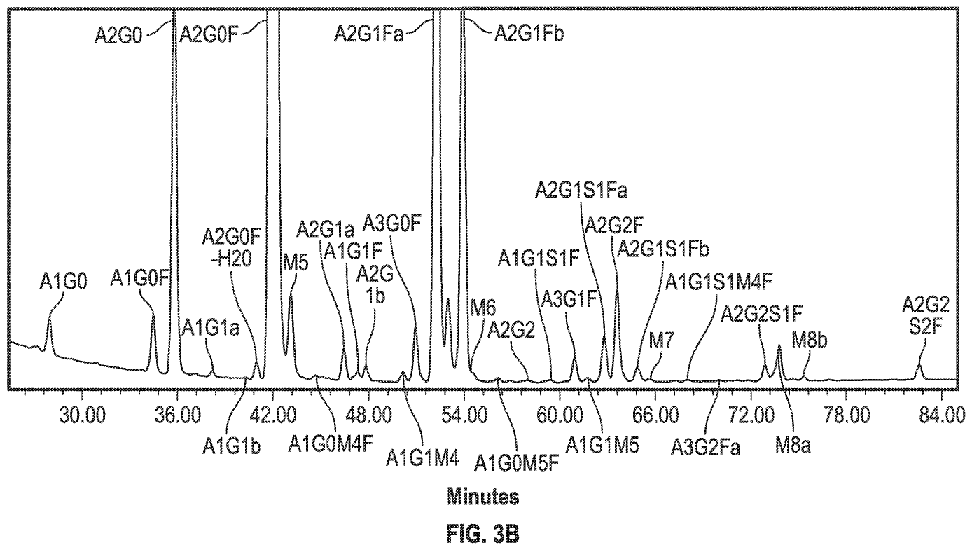

Various methods may be used for assessing glycans present in a glycoprotein-containing composition or for determining, detecting or measuring a glycoform profile (e.g., a glycoprofile) of a particular sample comprising glycoproteins. Suitable methods include, but are not limited to, positive ion MALDI-TOF analysis, negative ion MALDI-TOF analysis, weak anion exchange (WAX) chromatography, normal phase chromatography (NP-HPLC), exoglycosidase digestion, Bio-Gel P-4 chromatography, anion-exchange chromatography and one-dimensional n.m.r. spectroscopy, and combinations thereof. See, e.g., Mattu et al., JBC 273: 2260-2272 (1998); Field et al., Biochem J 299(Pt 1): 261-275 (1994); Yoo et al., MAbs 2(3): 320-334 (2010) Wuhrer M. et al., Journal of Chromatography B, 2005, Vol. 825, Issue 2, pages 124-133; Ruhaak L. R., Anal Bioanal Chem, 2010, Vol. 397:3457-3481 and Geoffrey, R. G. et. al. Analytical Biochemistry 1996, Vol. 240, pages 210-226. Also, a suitable method of assessing glycans present in a glycoprotein-containing composition may comprise enzymatic cleavage of glycans attached to the glycoprotein. The cleaved or released glycans are subsequently separated by Hydrophilic Interaction Liquid Chromatography (HILIC) and a chromatogram with several peaks is produced. Each peak of the chromatogram represents a distribution (amount or abundance) of a different glycan. Two views of a representative HILIC chromatogram comprising peaks for different glycans are provided in FIGS. 3A and 3B. For these purposes, % Peak Area=Peak Area/Total Peak Area×100%. Accordingly, the level of a particular glycan (or groups of glycans) is reported as a %. For example, if an antibody composition is characterized as having a Man6 level of 30%, it is meant that 30% of all glycans cleaved from the antibodies of the composition are Man6. As described in more detail herein, it is noted that such methods that remove glycans from the glycoproteins identify a distribution of glycan content for a glycoprotein, but do not provide information relating to paired glycans and/or unpaired glycans.

The present disclosure references high mannose glycans and afucosylated glycans of an antibody composition (see FIG. 1B for examples). As used herein, the term “high mannose glycans” or “HM glycans” encompasses glycans comprising 5, 6, 7, 8, or 9 mannose residues, abbreviated as Man5 or M5, Man6 or M6, Man7 or M7, Man8 or M8, and Man9 or M9, respectively. A level of HM glycans, in various aspects, is obtained by summing the % Man5, the % Man6, the % Man7, the % Man8, and the % Man9. As used herein, the term “afucosylated glycan” or “AF glycan” refers to glycans which lack a core fucose, e.g., an α1,6-linked fucose on the GlcNAc residue involved in the amide bond with the Asn of the N-glycosylation site. Afucosylated glycans include, but are not limited to, A1G0, A1G1, A2G0, A2G1 (a and b), A2G32, A1G1M4 and A1G1M5. It is noted that high mannose glycans also lack core fucose (and thus represent a subset of afucosylated glycans), but high mannose glycans have certain characteristics and may be referred to as a separate glycan group. Accordingly, unless explicitly stated otherwise, high mannose is understood to represent a separate characteristic and may be classified separately from, or as an additional characteristic of afucosylated glycans. See, e.g., Reusch and Tejada, Glycobiology 25(12): 1325-1334 (2015). A level of afucosylated glycans, in various aspects, is obtained by summing the % AlG0, the % A2G0, the % A2G1a, the % A2G1b, the % A2G2, the % A1G1M5, the % A1G1a.

The level (e.g., amount, abundance) of glycans (e.g., % HM glycans, % AF glycans) may be determined (e.g., measured) by any of the various methods known in the art for assessing glycans present in a glycoprotein-containing composition or for determining, detecting or measuring a glycoform profile (e.g., a glycoprofile) of a particular sample comprising glycoproteins. The level (e.g., amount, abundance) of glycans (e.g., % HM glycans, % AF glycans) of an antibody composition may be determined by measuring the level (e.g., amount, abundance) of such glycans in a sample of the antibody composition though a chromatography-based method, e.g., HILIC, and the level (e.g., amount, abundance) of glycans is expressed as a %, as described herein. The level of glycans of an antibody composition may be expressed as a % of all glycans cleaved from the antibodies of the composition. The level (e.g., amount, abundance) of glycans (e.g., % HM glycans, % AF glycans) may be determined (e.g., measured) by measuring the level of such glycans in a sample of the antibody composition. Samples of an antibody composition may be taken and the level (e.g., amount, abundance) of glycans (e.g., % HM glycans, % AF glycans) for each sample may be determined (e.g., measured). The % HM glycans and/or % AF glycans may be determined.

### Glycan Pairing and Methods of Measuring Unpaired Glycans of an Antibody Composition

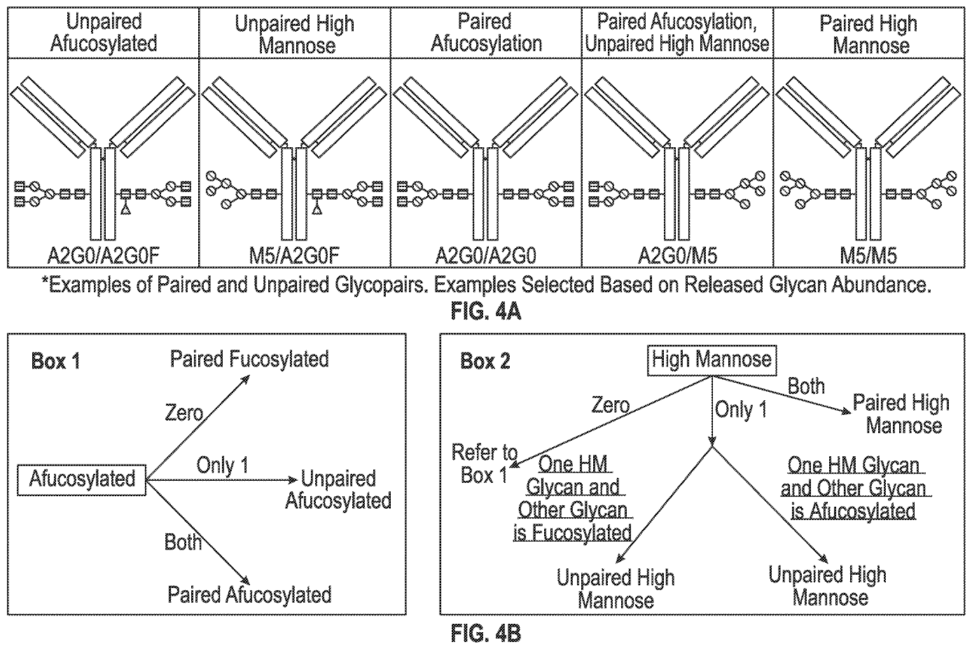

In exemplary instances of the present disclosure, the antibody composition comprises antibodies, each comprising two heavy chains and two light chains. In exemplary aspects, each of the antibodies of the antibody composition comprises a fragment crystallizable (Fc) region and two Fab regions. The Fc region of the antibody comprises two identical protein fragments derived from constant domains of the antibody heavy chains, and each protein fragment is glycosylated, e.g., comprises a glycan. Accordingly, in exemplary aspects, the Fc region of the antibody comprises a pair of glycans, or a glycan pair (also referred to herein as “glycopair”). In various aspects, the two glycans of the glycan pair are structurally different from each other. Alternatively, the glycans of the glycan pair are structurally identical. The glycan pair may be classified based on the presence or absence of glycans of the glycan pair comprising a core fucose, as well as the presence or absence of glycans of the glycan pair comprising high mannose. FIG. 4A is a series of drawings exemplifying an unpaired afucosylated antibody, a paired afucosylated antibody, an unpaired high mannose antibody and a paired high mannose antibody. FIG. 4B provides exemplary flow charts useful for assigning nomenclature to a glycan pair comprising canonical glycans. Exemplary paired high mannose glycans include glycans having (a) two identical high mannose glycans (e.g., high mannose of identical structure), such as Man5, Man6, Man7, Man8 or Man9, or (b) two non-identical high mannose glycans (e.g., high mannose glycans having different structures) but each high mannose glycan comprises Man5, Man6, Man7, Man8 or Man9 (e.g., Man5 as one glycan and Man6, Man7, Man8, or Man9 as the other glycan, or Man6 as one glycan and Man7, Man8, or Man9 as the other glycan, or Man7 as one glycan and Man8 or Man9 as the other glycan, or Man8 as one glycan and Man9 as the other glycan). Exemplary unpaired high mannose glycans include glycans having Man5, Man6, Man7, Man8 or Man9 as one glycan and a fucosylated or afucosylated glycan as the other glycan. Exemplary unpaired afucosylated glycan pairs include, for instance AGO, A2G0, A2G1a, A2G1b, or A2G2 as one glycan and a fucosylated glycan as the other glycan. Exemplary paired afucosylated glycans include, for instance, two identical afucosylated glycans (e.g., A1G0/A1G0) or two non-identical afucosylated glycans (e.g., A1G0/A2G2). Paired afucosylated glycans include, for example, (i) AlG0 as one glycan and A2G0, A2G1a, A2G1b, or A2G2 or (ii) A2G0 on one chain and A2G1a, A2G1b, or A2G2 on the other Fc chain or (iii) A2G1a on one Fc chain and A2G1b or A2G2 on the other Fc chain or (iv) A2G1b on one Fc chain and A2G2 on the other chain. Paired afucosylated glycans can include a high mannose as one glycan and any of A1G0, A2G0, A2G1a, A2G1b, or A2G2 as the other, e.g., M5/A1G0. From the high mannose perspective, if only one glycan of the pair comprises a high mannose, the paired afucosylated glycan may be considered as an “unpaired high mannose”.

In exemplary instances, the glycan pair is given a designation based on the fucosylation/afucosylation status of each glycan of the glycan pair. In exemplary aspects, the glycan pair is given a designation based on the high mannose status of each glycan of the glycan pair. Suitable designations will be appreciated in the context of the glycan structures of interest in the particular scenario. In various aspects, the glycan pair is given two designations, wherein a first designation is based on the fucosylation/afucosylation status of each glycan of the glycan pair and a second designation is based on the high mannose status of each glycan of the glycan pair, and, optionally, a summary designation, which is used for quantifying the abundance of Fc fragments comprising (i) paired afucosylated glycans, (ii) unpaired afucosylated glycans, (iii) paired high mannose glycans, and/or (iv) unpaired high mannose glycans, is assigned. Table A below provides an exemplary way of assigning these designations. In various instances, a Summary Designation is assigned by applying three rules relating to the First Designation (Designation 1) and Second Designation (Designation 2), as follows: (1) when Designation 1 is paired fucosylated, the Summary Designation is paired fucosylated (regardless of Designation 2); (2) when Designation 2 is no HM, the Summary Designation is the same as Designation 1; and (3) when Designation 2 is paired HM or unpaired HM and Designation 1 is not paired fucosylated, the Summary Designation is the same as Designation 2 (e.g., Scenarios 4 and 10) or is a combination of Designation 1 and Designation 2 (e.g., Scenarios 5, 8, and 9).

TABLE A

| | Fc | | | | | Summary |
|---|---|---|---|---|---|---|
| Scenario | Chain | HM | Fuc/AF | Designation 1 | Designation 2 | Designation |
| 1 | 1 | negative | Fuc | paired fucosylated | no HM | paired fucosylated |
| | 2 | negative | Fuc | | | |
| 2 | 1 | negative | AF | paired AF | no HM | paired AF |
| | 2 | negative | AF | | | |
| 3 | 1 | negative | Fuc | unpaired AF | no HM | unpaired AF |
| | 2 | negative | AF | | | |
| 4 | 1 | positive | AF | paired AF | paired HM | paired HM |
| | 2 | positive | AF | | | |
| 5* | 1 | positive | AF | unpaired AF | paired HM | paired HM, |
| | 2 | positive | Fuc | | | unpaired AF |
| 6 | 1 | positive | Fuc | paired fucosylated | paired HM | paired fucosylated |
| | 2 | positive | Fuc | | | |
| 7 | 1 | positive | Fuc | paired fucosylated | unpaired HM | paired fucosylated |
| | 2 | negative | Fuc | | | |
| 8 | 1 | positive | AF | paired AF | unpaired HM | unpaired HM, |
| | 2 | negative | AF | | | paired AF |
| 9* | 1 | positive | Fuc | unpaired AF | unpaired HM | unpaired HM, |
| | 2 | negative | AF | | | unpaired AF |
| 10 | 1 | positive | AF | unpaired AF | unpaired HM | unpaired HM |
| | 2 | negative | Fuc | | | |

Fuc, fucosylated; AF, afucosylated; HM, high mannose. Designation 1 is based on fucosylated/afucosylated status of each glycan on each Fc chain. Designation 2 is based on the high mannose status of each glycan on each Fc chain. Summary designation is based on 3 rules: (1) when Designation 1 is paired fucosylated, the Summary Designation is paired fucosylated (regardless of Designation 2); (2) when Designation 2 is no HM, the Summary Designation is the same as Designation 1; and (3) when Designation 2 is paired HM or unpaired HM and Designation 1 is not paired fucosylated, the Summary Designation is the same as Designation 2 (e.g., Scenarios 4 and 10) or is a combination of Designations 1 and 2 (e.g., Scenarios 5, 8 and 9).
*Scenarios 5 and 9, involving a fucosylated glycan comprising a high mannose, are uncommon occurrences.

In various instances, the antibody, or its Fc region, comprising the glycan pair may be described in terms of its glycan pair classification. Thus, an antibody or an Fc region may be described as “paired afucosylated”, “unpaired afucosylated”, “paired fucosylated”, “paired high mannose” or “unpaired high mannose” by virtue of its glycan pair classification. Similarly, the antibody or its Fc region may be described as comprising paired afucosylated glycans (or paired afucosylated glycan pairs), unpaired afucosylated glycans (or unpaired afucosylated glycan pairs), paired high mannose glycans (paired high mannose glycan pairs) and/or unpaired high mannose glycans (unpaired high mannose glycan pairs) by virtue of its glycan pair classification. As discussed below, antibodies may be cleaved into fragments. Fc fragments comprise the Fc region of an antibody and may be described as “paired afucosylated”, “unpaired afucosylated”, “paired fucosylated”, “paired high mannose” or “unpaired high mannose” by virtue of the glycan pair classification of the Fc region.

An antibody composition may be characterized in terms of its paired glycan content and/or its unpaired glycan content. For example, an antibody composition may be characterized in terms of its paired afucosylated glycan content and/or unpaired unfucosylated glycan content and/or its paired high mannose content and/or unpaired high mannose content. The abundance (e.g., paired afucosylated glycan content and/or unpaired unfucosylated glycan content and/or its paired high mannose content and/or unpaired high mannose content) as described herein may be referred to as relative or absolute abundance. In exemplary instances, the absolute content of glycans may be expressed in units measuring levels of the glycans themselves, for example in terms of mass, moles, mass or molar units per volume unit, arbitrary units, area under the curve, or intensities of mass spectral peaks, e.g., deconvoluted mass spectral peaks (e.g., as may be determined from a chromatograph). In exemplary instances, an antibody composition is characterized in terms of its relative abundance of unpaired glycans, meaning that the amount of unpaired glycans is expressed as an amount relative to the sum of paired glycans and unpaired glycans of the antibody composition. In exemplary aspects, the antibody composition is characterized in terms of its relative abundance of unpaired afucosylated glycans. In exemplary aspects, the antibody composition is characterized in terms of its relative abundance of unpaired high mannose glycans.

The term “relative abundance of unpaired afucosylated glycans” which is synonymous with “relative unpaired afucosylated glycan content”, “relative abundance of unpaired afucosylated glycan pairs” and “relative % unpaired afucosylated glycans” is calculated as dividing the percentage of unpaired afucosylated glycans by the sum of the percentage of unpaired afucosylated glycans and the percentage of paired afucosylated glycans) and multiplying by 100%.

The term “relative abundance of unpaired high mannose glycans” which is synonymous with “relative unpaired high mannose glycan content”, “relative abundance of unpaired high mannose glycan pairs” and “relative % unpaired high mannose glycans” is calculated as the percentage of unpaired high mannose glycans divided by the sum of the percentage of unpaired high mannose glycans and the percentage of paired high mannose glycans) multiplied by 100%.

The term “relative abundance of paired afucosylated glycans” is synonymous with “relative paired afucosylated glycan content” “relative abundance of paired afucosylated glycan pairs” and “relative % paired afucosylated glycans” is calculated as dividing the percentage of paired afucosylated glycans by the sum of the percentage of unpaired afucosylated glycans and the percentage of paired afucosylated glycans) and multiplying by 100%.

The term “relative abundance of paired high mannose glycans” is synonymous with “relative paired high mannose glycan content”, “relative abundance of paired high mannose glycan pairs” and “relative % paired high mannose glycans” is calculated as dividing the percentage of paired high mannose glycans by the sum of the percentage of unpaired high mannose glycans and the percentage of paired high mannose glycans) and multiplying by 100%.

In various aspects of the present disclosure, the sum of the relative % unpaired afucosylated glycans and the relative % paired afucosylated glycans equals 100%. Accordingly, in various aspects, if the relative % paired afucosylated glycans is known, the relative % unpaired afucosylated glycans may be determined (e.g., calculated) by subtracting the relative % paired afucosylated glycans from 100%. Also, in various instances, if the relative % unpaired afucosylated glycans is known, the relative % paired afucosylated glycans may be determined (e.g., calculated) by subtracting the relative % unpaired afucosylated glycans from 100%. In various aspects of the present disclosure, the sum of the relative % unpaired high mannose glycans and the relative % paired high mannose glycans equals 100%. Accordingly, in various aspects, if the relative % paired high mannose glycans is known, the relative 5% unpaired high mannose glycans may be determined (e.g., calculated) by subtracting the relative % paired high mannose glycans from 100%. Also, in various instances, if the relative % unpaired high mannose glycans is known, the relative % paired high mannose glycans may be determined (e.g., calculated) by subtracting the relative % unpaired high mannose glycans from 100%.

### Methods of Determining Relative Unpaired Glycan Content

The present disclosure provides methods of determining the relative unpaired glycan content of an IgG antibody composition. In exemplary embodiments, the method comprises (a) treating the IgG antibody composition with two enzymes to form a mixture of Fab fragments and Fc fragments, wherein one enzyme cleaves an antibody heavy chain at a site N-terminal to the hinge region disulfide linkages and the other enzyme cleaves the β1,4 linkage between core GlcNAc residues to form Fc fragments each comprising a pair of core glycan structures, (b) separating the Fab fragments from the Fc fragments, and (c) quantifying the abundance of Fc fragments comprising (i) paired afucosylated glycans, (ii) unpaired afucosylated glycans, (iii) paired high mannose glycans, and/or (iv) unpaired high mannose glycans, to determine the relative unpaired afucosylated (AF) glycan content and relative unpaired high mannose (HM) glycan content. In (c), the abundance of (i) paired afucosylated Fc fragments, (ii) unpaired afucosylated Fc fragments, (iii) paired high mannose Fc fragments, and/or (iv) unpaired high mannose Fc fragments is/are quantified. In exemplary aspects, the relative abundance of (i) paired afucosylated Fc fragments, (ii) unpaired afucosylated Fc fragments, (iii) paired high mannose Fc fragments, and/or (iv) unpaired high mannose Fc fragments is/are quantified. In exemplary instances, the relative abundance of unpaired afucosylated glycans and the relative abundance of unpaired high mannose glycans is determined. In exemplary aspects the one enzyme and the other enzyme are incubated simultaneously or concurrently with the IgG antibody composition. For example, the IgG antibody composition may be incubated with the one enzyme (that cleaves an antibody heavy chain at a site N-terminal), and the other enzyme (that cleaves the β1,4 linkage between core GlcNAc residues) may be subsequently added. In exemplary aspects, the one enzyme and the other enzyme are incubated sequentially. For example, the antibody IgG antibody composition may be incubated with the one enzyme (that cleaves an antibody heavy chain at a site N-terminal), and after that incubation is over, the IgG antibody composition may be incubated with the other enzyme (that cleaves the β1,4 linkage between core GlcNAc residues).

In various aspects of the presently disclosed method of determining the relative unpaired glycan content, the one enzyme which cleaves an antibody heavy chain at a site N-terminal to the hinge region disulfide linkages is a cysteine protease. In various instances, the enzyme is a member of the IgdE protease family, optionally, an IgdE expressed by a Streptococcus. In various instances, the enzyme is structurally identical or highly similar to an IgdE protease expressed by Streptococcus agalactiae. In various aspects, the enzyme is structurally identical or highly similar to an enzyme expressed by a Porphyromonas anaerobe. In various instances, the enzyme is structurally identical or highly similar to an enzyme expressed by a Porphyromonas gingivalis. In various instances, the site is between Thr and His or between Lys and Thr of the sequence KTHTCPP (SEQ ID NO: 1) of an IgG1 antibody heavy chain. In exemplary aspects, the method comprises treating the IgG antibody composition with the cysteine protease for at least 8 hours or at least 12 hours, e.g., at least 9 hours, at least 10 hours, at least 11 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours.

In various aspects, the other enzyme which cleaves the β1,4 linkage between core GlcNAc residues is an IgG-specific enzyme. In exemplary aspects, the IgG-specific enzyme is an endoglycosidase, e.g., an Endoglycosidase D, an Endoglycosidase F, an Endoglycosidase F1, an Endoglycosidase F2, an Endoglycosidase H, or an Endo S or EndoS2 endoglycosidase. In exemplary instances, the endoglycosidase is an Endoglycosidase S. In exemplary aspects, the method comprises treating the IgG antibody composition with the IgG-specific enzyme for less than 1 hour, e.g., less than 55 minutes, less than 50 minutes, less than 45 minutes, less than 40 minutes, less than 35 minutes, less than 30 minutes. In various aspects, the method comprises treating the IgG antibody composition with the IgG-specific enzyme for about 30 minutes. In various aspects, the other enzyme is added to the IgG composition during its the incubation with the enzyme of the IgdE protease family (e.g., for the last 30 minutes to an hour of the incubation, as applicable). In various aspects, the other enzyme is added to the IgG composition after the incubation with the enzyme of the IgdE protease family has been completed.

In exemplary aspects, the method comprises separating the Fab fragments from the Fc fragments of the IgG antibody of the composition. Optionally, the method comprises separating the Fab fragments from the Fc fragments by a chromatography. The chromatography may be any suitable type which can separate the Fab fragments from the Fc fragments, including, but not limited to, affinity chromatography (e.g., Protein A chromatography), anion or cation exchange chromatography, liquid chromatography (LC, e.g., reversed phase LC, HILIC), and the like. Optionally, the chromatography is a reversed phase liquid chromatography. In various aspects, spectral peaks of the Fc fragments elute as one chromatographic peak which is selected for analysis of the mass spectrometry data. In exemplary aspects, the method comprises performing mass spectroscopy data analysis of the Fc fragments to obtain one or more mass spectral peaks. In various aspects, the method comprises deconvoluting the mass spectral peaks to obtain deconvoluted mass spectral peaks. Suitable methods of deconvoluting mass spectral peaks are known in the art. See, e.g., Gadgil et al., J. Am. Soc. Mass Spectrom. 17, 867 (2006); Murray et al., Pure and Applied Chemistry, vol. 85, no. 7, 2013, pp. 1515-1609; Marchetti and Mignerey, Nuclear Instruments and Methods in Physics Research Section A: Accelerators, Spectrometers, Detectors and Associated Equipment 324(1-2): 288-296 (1993); Xu et al., Rapid Commun Mass Spectrom. 2018 May 30; 32(10):763-774. In exemplary instances, the method comprises matching the molecular weight of each deconvoluted mass spectral peak against a database of glycan pairs and the associated molecular weight to identify the glycan pair. In exemplary instances, the method comprises quantifying the abundance of Fc fragments comprising (i) paired afucosylated glycans, (ii) unpaired afucosylated glycans, (iii) paired high mannose glycans, and/or (iv) unpaired high mannose glycans, to determine the relative unpaired afucosylated (AF) glycan content and relative unpaired high mannose (HM) glycan content. In various aspects, the abundance of Fc fragments comprising paired afucosylated glycans and the abundance of Fc fragments comprising unpaired afucosylated glycans are summed so that relative unpaired afucosylated glycans may be determined. In various aspects, the abundance of Fc fragments comprising paired high mannose glycans and the abundance of Fc fragments comprising unpaired high mannose glycans are summed so that relative unpaired high mannose glycans may be determined. Suitable methods for carrying out the method of the present disclosure is described in detail in Example 1. The database in exemplary aspects comprises the glycan pairs and their associated theoretical molecular weights of Table 1 of Example 1. In various aspects, the separating and quantifying of the presently disclosed methods occur in less than 2 hours, optionally, less than 95 minutes, less than 85 minutes, less than 75 minutes, less than 60 minutes, less than 45 minutes, less than 30 minutes or less than 20 minutes (optionally, about 19 minutes, about 18 minutes, about 17 minutes, about 16 minutes, about 15 minutes, about 14 minutes, about 13 minutes, about 12 minutes, about 11 minutes, about 10 minutes, about 9 minutes, about 8 minutes, about 7 minutes, about 6 minutes, about 5 minutes). The presently disclosed methods in exemplary aspects comprise separating and quantifying in less than 30 minutes or less than 20 minutes. In various aspects, the separating and quantifying of the presently disclosed methods occur in about 5 minutes to about 25 minutes, about 5 minutes to about 20 minutes, about 5 minutes to about 15 minutes, about 5 minutes to about 10 minutes, about 10 minutes to about 25 minutes, about 15 minutes to about 25 minutes, about 20 minutes to about 25 minutes). The presently disclosed methods of determining the relative unpaired glycan content advantageously require less time than prior methods.

### Applications of Determining Relative Unpaired Glycon Content

The present inventive methods of determining relative unpaired glycans are useful during the development and/or production of an antibody composition. For example, the method of determining relative unpaired glycans may be used for analyzing an IgG antibody composition. Accordingly, methods of analyzing an IgG antibody composition are provided herein. In exemplary embodiments, the method comprises determining the relative unpaired glycan content of a sample of the IgG antibody composition in accordance with any one of the presently disclosed methods of determining the relative unpaired glycan content. In various aspects, the method comprises comparing the relative unpaired AF glycan content and/or relative unpaired HM glycan content of the IgG antibody composition to the relative unpaired AF glycan content and/or relative unpaired HM glycan content of a reference product. The method may be performed in manufacturing, for example to determine if a lot of the IgG antibody composition meets a specification.

Additionally provided are methods of monitoring production of an IgG antibody composition. In exemplary embodiments, the method comprises determining the relative unpaired glycan content of an IgG antibody composition in accordance with any one of the presently disclosed methods of determining the relative unpaired glycan content, for a first sample obtained at a first timepoint and for a second sample taken at a second timepoint which is different from the first timepoint. In exemplary aspects, the method comprises comparing the relative unpaired AF glycan content and/or relative unpaired HM glycan content of the first sample to the relative unpaired AF glycan content and/or relative unpaired HM glycan content of the second sample.

In various aspects, each of the first sample and second sample is a sample of in-process material. In various instances, the first sample is a sample of in-process material and the second sample is a sample of a manufacturing lot. Optionally, the first sample is a sample obtained before one or more conditions of the cell culture are modified and the second sample is a sample obtained after the one or more conditions of the cell culture are modified.

In various aspects, the presently disclosed methods are useful for determining whether an IgG antibody composition meets the statistical criteria which includes appropriate acceptance levels and/or appropriate rejection levels.

In various aspects, the methods of analyzing an IgG antibody composition and/or methods of monitoring production of an IgG antibody composition occur during the development or production of an antibody composition. Accordingly, methods of producing an antibody composition are also provided by the present disclosure. In exemplary embodiments, the method comprises (A) determining the relative unpaired glycan content of a sample of an IgG antibody composition, wherein relative unpaired glycan content of the sample of the antibody composition is determined in accordance with any one of the presently disclosed methods of determining the relative unpaired glycan content, wherein the sample is a sample of in-process material, wherein, when the relative unpaired glycan content is determined as outside a predetermined target range, the method further comprises (B) modifying one or more conditions of the cell culture to obtain a modified cell culture and determining the relative unpaired glycan content, and optionally, repeating (A) and (B) until the relative unpaired glycan content is within the predetermined target range. In various instances, one or more conditions of the cell culture are modified to primarily change the relative unpaired HM glycan content to achieve the target range of relative unpaired glycan content. In various aspects, one or more conditions of the cell culture are modified to primarily change the relative unpaired AF glycan content to achieve the target range of relative unpaired glycan content. Additional embodiments of methods of producing antibody compositions are further provided below. See, e.g., Methods of Producing Antibody Compositions.

### ADCC and Methods of Modifying ADCC Activity Levels

The data presented herein support that the relative unpaired glycan content of an antibody composition is related to the ADCC activity level for the antibody composition and that the ADCC activity level of the antibody composition may be modified by modifying the relative unpaired glycan content of the antibody composition. Without being bound to a particular theory, the relative unpaired afucosylated glycan content and/or the relative unpaired high mannose glycan content of an antibody composition is related to the ADCC activity level of the antibody composition, and, changing the relative unpaired afucosylated glycan content and/or the relative unpaired high mannose glycan content of the antibody composition leads to changing the ADCC activity level of the antibody composition, it is further contemplated that relative unpaired glycan content (e.g., relative unpaired afucosylated glycan content and/or relative unpaired high mannose glycan content) has greater leverage on ADCC than the relative paired glycan content (e.g., relative paired afucosylated glycan content and/or relative paired high mannose glycan content), so that a percent change in the relative unpaired glycan content will have a greater effect on ADCC than the same percent change in relative paired glycan content. Accordingly, provided herein are methods of modifying the ADCC level of an antibody composition. In exemplary embodiments, the method comprises modifying the relative unpaired afucosylated glycan content of an antibody composition and/or the relative unpaired high mannose glycan content of an antibody composition.

The term “ADCC” or “antibody-dependent cell-mediated cytotoxicity” or “antibody-dependent cellular cytotoxicity” refers to the mechanism by which an effector cell of the immune system (e.g., natural killer cells (NK cells), macrophages, neutrophils, eosinophils) actively lyses a target cell, whose membrane-surface antigens have been bound by specific antibodies. ADCC is a part of the adaptive immune response and occurs when antigen-specific antibodies bind to (1) the membrane-surface antigens on a target cell through its antigen-binding regions and (2) to Fc receptors on the surface of the effector cells through its Fc region. Binding of the Fc region of the antibody to the Fc receptor causes the effector cells to release cytotoxic factors that lead to death of the target cell (e.g., through cell lysis or cellular degranulation).

Fc receptors are receptors on the surfaces of B lymphocytes, follicular dendritic cells, NK cells, macrophages, neutrophils, eosinophils, basophils, platelets and mast cells that bind to the Fc region of an antibody. Fc receptors are grouped into different classes based on the type of antibody that they bind. For example, an Fcγ receptor is a receptor for the Fc region of an IgG antibody, an Fc-alpha receptor is a receptor for the Fc region of an IgA antibody, and an Fc-epsilon receptor is a receptor for the Fc region of an IgE antibody.

The term “FcγR” or “Fc-gamma receptor” is a protein belonging to the IgG superfamily involved in inducing phagocytosis of opsonized cells or microbes. See, e.g., Fridman W H. Fc receptors and immunoglobulin binding factors. FASEB Journal. 5 (12): 2684-90 (1991). Members of the Fc-gamma receptor family include: FcγRI (CD64), FcγRIIA (CD32), FcγRIIB (CD32), FcγRIIIA (CD16a), and FcγRIIIB (CD16b). The sequences of FcγRI, FcγRIIA, FcγRIIB, FcγRIIIA, and FcγRIIIB can be found in many sequence databases, for example, at the Uniprot database (www.uniprot.org) under accession numbers P12314 (FCGR1_HUMAN), P12318 (FCG2A_HUMAN), P31994 (FCG2B_HUMAN), P08637 (FCG3A_HUMAN), and P08637 (FCG3A_HUMAN), respectively.

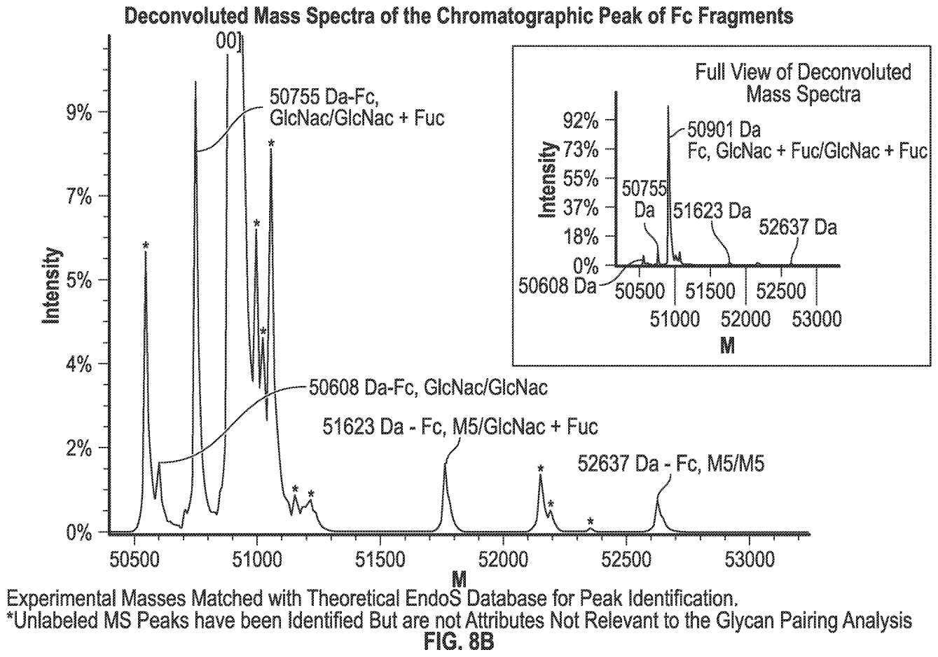

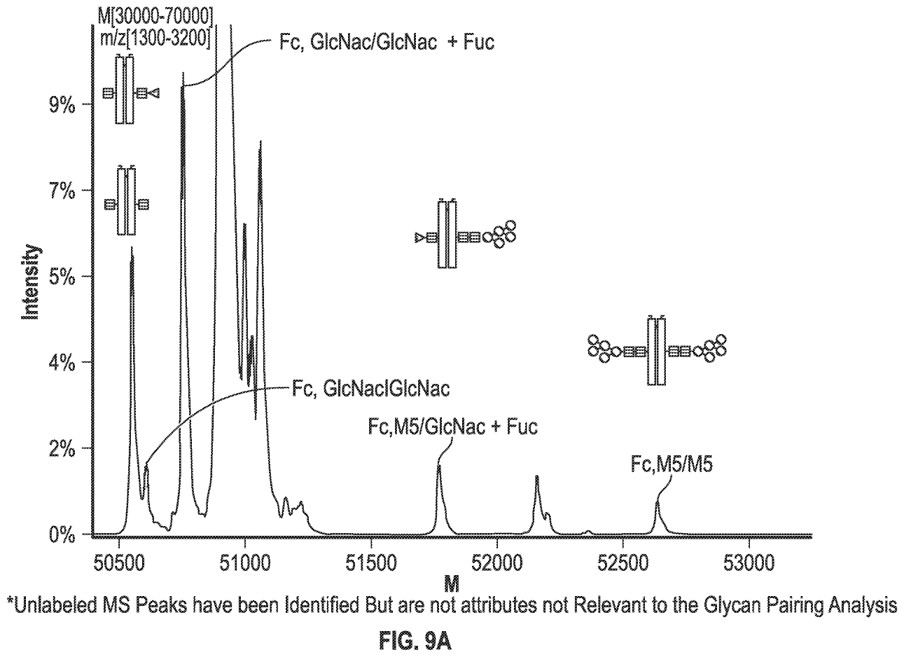

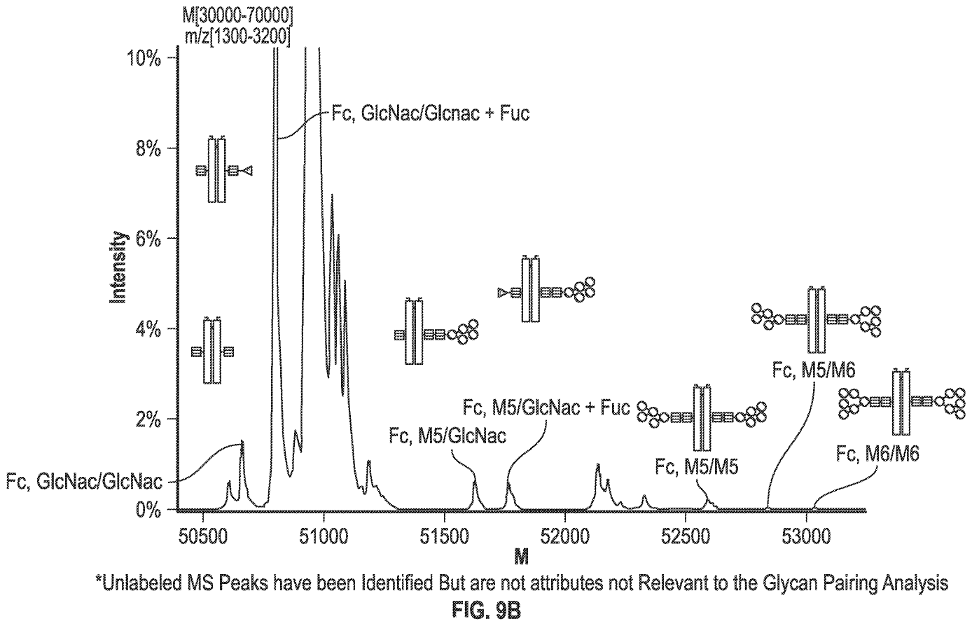

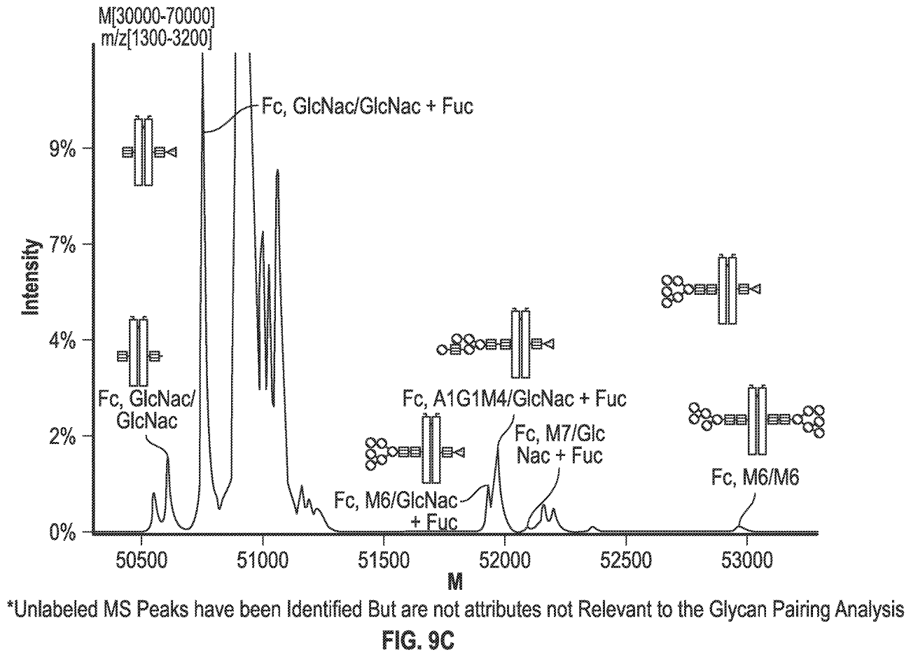

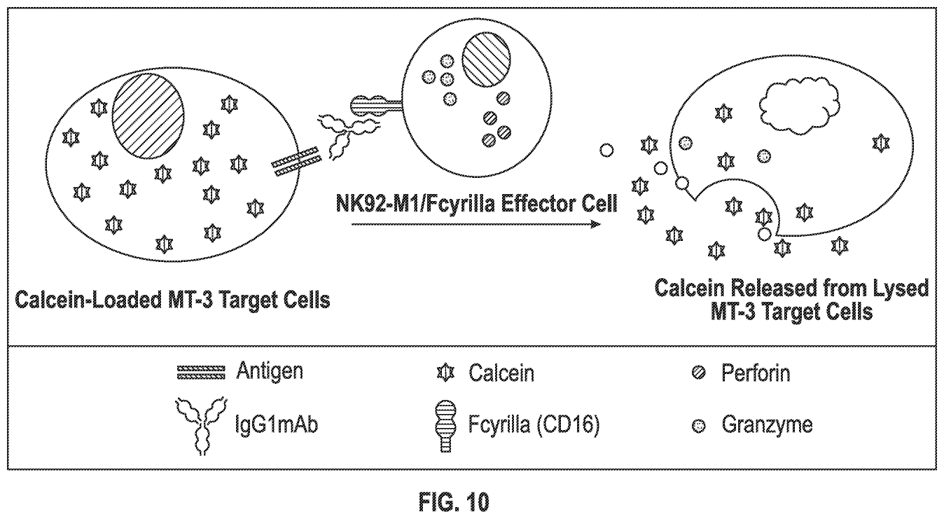

The term “ADCC activity” or “ADCC level” refers to the extent to which ADCC is activated or stimulated. Methods of measuring or determining the ADCC level of an antibody composition, including commercially available assays and kits for measuring or determining the ADCC level, are well-known in the art, as described, Yamashita et al., Scientific Reports 6: article number 19772 (2016), doi:10.1038/srep19772); Kantakamalakul et al., “A novel EGFP-CEM-NKr flow cytometric method for measuring antibody dependent cell mediated-cytotoxicity (ADCC) activity in HIV-1 infected individuals”, J Immunol Methods 315 (Issues 1-2): 1-10; (2006); Gomez-Roman et al., “A simplified method for the rapid fluorometric assessment of antibody-dependent cell-mediated cytotoxicity”, J Immunol Methods 308 (Issues 1-2): 53-67 (2006); Schnueriger et al., Development of a quantitative, cell-line based assay to measure ADCC activity mediated by therapeutic antibodies”, Molec Immunology 38 (Issues 12-13): 1512-1517 (2011); and Mata et al., “Effects of cryopreservation on effector cells for antibody dependent cell-mediated cytotoxicity (ADCC) and natural killer (NK) cell activity in “Cr-release and CD107a assays”, J Immunol Methods 406: 1-9 (201.4); all herein incorporated by reference for all purposes. The term “ADCC Assay” or “FcγR reporter gene assay” refers to an assay, kit or method useful to determine the ADCC activity of an antibody. Exemplary methods of measuring or determining the ADCC activity of an antibody in the methods described herein include the ADCC assay described in the Example 3 or the ADCC Reporter assay commercially available from Promega (Catalog No. G7010 and G7018). In some embodiments, ADCC activity is measured or determined using a calcein release assay containing one or more of the following: a FcγRIIIa (158V)-expressing NK92(M1) cells as effector cells and HCC2218 cells or MT-3 cells as target cells labeled with calcein-AM. An illustration of an exemplary calcein release assay is provided as FIG. 10. In exemplary aspects of the calcein release assay, a standard curve is created using various concentrations of a reference antibody (FIG. 10B).

In exemplary aspects, the level of ADCC of an antibody composition is determined by a quantitative cell based assay which measures the ability of the antibodies of the antibody composition to mediate cell cytotoxicity in a dose dependent manner in cells expressing the antigen of the antibodies and engaging FcγRIIIA receptors on effector cells through the Fc domain of the antibodies. In various embodiments, the method comprises the use of target cells harboring detectable labels that are released when the target cells are lysed by the effector cells. The amount of detectable label released from the target cells is a measure of the ADCC activity of the antibody composition. The amount of detectable label released from the target cells, in some aspects, is compared to a baseline. Also, the ADCC level may be reported as a % ADCC relative to a control % ADCC. In various aspects, the % ADCC is a relative % ADCC, which optionally, is relative to a control % ADCC. In various aspects, the control % ADCC is the % ADCC of a reference antibody. In exemplary instances, the control % ADCC is within a range of about 60% to about 130%. Optionally, the % ADCC is determined by the assay described in Example 3.

In exemplary embodiments, the method of modifying (increasing or decreasing) the ADCC level of an antibody composition comprises modifying (increasing or decreasing) the relative unpaired afucosylated glycan content of an antibody composition and/or the relative unpaired high mannose glycan content of an antibody composition. In exemplary aspects, the presently disclosed method of modifying the ADCC level of an antibody composition comprises increasing the relative unpaired afucosylated glycan content to increase the level of ADCC activity. In exemplary instances, the method of modifying the ADCC level of an antibody composition comprises increasing the relative unpaired high mannose glycan content to increase the level of ADCC activity. In various aspects, the increase in ADCC activity level provided by the methods of the disclosure is at least or about a 1% to about a 20% increase (e.g., at least or about a 1% increase, at least or about a 2% increase, at least or about a 3% increase, at least or about a 4% increase, at least or about a 5% increase, at least or about a 6% increase, at least or about a 7% increase, at least or about a 8% increase, at least or about a 9% increase, at least or about a 10% increase, at least or about a 11% increase, at least or about a 12% increase, at least or about a 13% increase, at least or about a 14% increase, at least or about a 15% increase, at least or about a 16% increase, at least or about a 17% increase, at least or about a 18% increase, at least or about a 19% increase, at least or about a 20% increase) relative to a control. A suitable control may be the same protein or antibody composition without the increase in the relative unpaired glycan content. In exemplary embodiments, the increase in ADCC activity level provided by the methods of the disclosure is about 10% to about 100%, optionally, about 10% to about 90%, about 10% to about 80%, about 10% to about 70%, about 10% to about 70%, about 10% to about 50%, about 10% to about 40%, about 10% to about 30%, about 10% to about 20%, about 10% to about 15%, about 20% to about 100%, about 30% to about 100%, about 40% to about 100%, about 50% to about 100%, about 60% to about 100%, about 70% to about 100%, about 80% to about 100%, about 90% to about 100%, or about 95% to about 100%. The increase can be relative to the control. In exemplary embodiments, the increase in ADCC activity level provided by the methods of the disclosure is over 100%, e.g., 200%, 300%, 400%, 500%, 600%, 700%, 800%, 900% or even 1000% relative a control. In exemplary embodiments, the level of ADCC activity increases by at least about 1.5-fold, relative a control. A suitable control may be an ADCC activity level of the same protein or antibody composition without the change in the relative unpaired glycan content. In exemplary embodiments, the level of ADCC activity increases by at least about 2-fold, relative a control. In exemplary embodiments, the level of ADCC activity increases by at least about 3-fold, relative a control. In exemplary embodiments, the level of ADCC activity increases by at least about 4-fold or about 5-fold, relative to a control. In various aspects, the increase in the level of ADCC activity of the antibody composition is related to the increase in relative unpaired glycan content. For instance, the increase in the level of ADCC activity of the antibody composition is at least or about X % per ^{~}1% increase in relative unpaired glycan content, wherein X % is at least or about a 1% to about a 20% increase (e.g., at least or about a 1% increase, at least or about a 2% increase, at least or about a 3% increase, at least or about a 4% increase, at least or about a 5% increase, at least or about a 6% increase, at least or about a 7% increase, at least or about a 3% increase, at least or about a 9% increase, at least or about a 10% increase, at least or about a 11% increase, at least or about a 12% increase, at least or about a 13% increase, at least or about a 14% increase, at least or about a 15% increase, at least or about a 16% increase, at least or about a 17% increase, at least or about a 18% increase, at least or about a 19% increase, at least or about a 20% increase). Also, for example, X % may be about 10% to about 100%, optionally, about 10% to about 90%, about 10% to about 80%, about 10% to about 70%, about 10% to about 60%, about 10% to about 50%, about 10% to about 40%, about 10% to about 30%, about 10% to about 20%, about 10% to about 15%, about 20% to about 100%, about 30% to about 100%, about 40% to about 100%, about 50% to about 100%, about 60% to about 100%, about 70% to about 100%, about 80% to about 100%, about 90% to about 100%, or about 95% to about 100%.

In various aspects, the method of modifying the ADCC level of an antibody composition comprises decreasing the relative unpaired afucosylated glycan content to decrease the level of ADCC activity. In various instances, the method of modifying the ADCC level of an antibody composition comprises decreasing the relative unpaired high mannose glycan content to decrease the level of ADCC activity. In various aspects, the decrease in the ADCC activity level provided by the methods of the disclosure is at least or about a 1% to about a 20% decrease (e.g., at least or about a 1% decrease, at least or about a 2% decrease, at least or about a 3% decrease, at least or about a 4% decrease, at least or about a 5% decrease, at least or about a 6% decrease, at least or about a 7% decrease, at least or about a 8% increase, at least or about a 9% increase, at least or about a 10% increase, at least or about a 11% increase, at least or about a 12% increase, at least or about a 13% increase, at least or about a 14% increase, at least or about a 15% increase, at least or about a 16% increase, at least or about a 17% increase, at least or about a 18% increase, at least or about a 19% increase, at least or about a 20% increase) relative a control. A suitable control may be the same protein or antibody composition without the change in the relative unpaired glycan and overall glycan composition content. In exemplary embodiments, the decrease in the ADCC activity level provided by the methods of the disclosure is about 10% to about 100%, optionally, about 10% to about 90%, about 10% to about 80%, about 10% to about 70%, about 10% to about 60%, about 10% to about 50%, about 10% to about 40%, about 10% to about 30%, about 10% to about 20%, about 10% to about 15%, about 20% to about 100%, about 30% to about 100%, about 40% to about 100%, about 50% to about 100%, about 60% to about 100%, about 70% to about 100%, about 80% to about 100%, about 90% to about 100%, or about 95% to about 100%. The decrease can be relative to a control. In exemplary embodiments, the decrease in the ADCC activity level provided by the methods of the disclosure is over 100%, e.g., 200%, 300%, 400%, 500%, 600%, 700%, 800%, 900% or even 1000% relative a control. In exemplary embodiments, the level of ADCC activity decreases by at least about 1.5-fold, relative a control. A suitable control may be the ADCC activity level of the same protein or antibody composition without the change in the glycan content. In exemplary embodiments, the level of ADCC activity decreases by at least about 2-fold, relative a control. In exemplary embodiments, the level of ADCC activity decreases by at least about 3-fold, relative a control. In exemplary embodiments, the level of ADCC activity decreases by at least about 4-fold or about 5-fold, relative to a control. In various aspects, the decrease in the level of ADCC activity of the antibody composition is related to the decrease in relative unpaired glycan content. For instance, the decrease in the level of ADCC activity of the antibody composition is at least or about X % per ^{~}1% decrease in relative unpaired glycan content, wherein X % is at least or about a 1% to about a 20% decrease (e.g., at least or about a 1% decrease, at least or about a 2% decrease, at least or about a 3% decrease, at least or about a 4% decrease, at least or about a 5% decrease, at least or about a 6% decrease, at least or about a 7% decrease, at least or about a 8% increase, at least or about a 9% increase, at least or about a 10% increase, at least or about a 11% increase, at least or about a 12% increase, at least or about a 13% increase, at least or about a 14% increase, at least or about a 15% increase, at least or about a 16% increase, at least or about a 17% increase, at least or about a 18% increase, at least or about a 19% increase, at least or about a 20% increase). Also, for example, X % may be about 10% to about 100%, optionally, about 10% to about 90%, about 10% to about 80%, about 10% to about 70%, about 10% to about 60%, about 10% to about 50%, about 10% to about 40%, about 10% to about 30%, about 10% to about 20%, about 10% to about 15%, about 20% to about 100%, about 30% to about 100%, about 40% to about 100%, about 50% to about 100%, about 60% to about 100%, about 70% to about 100%, about 80% to about 100%, about 90% to about 100%, or about 95% to about 100%.

In exemplary aspects, the modification (increase or decrease) effected by the presently disclosed methods are relative to a “control”. In exemplary aspects, the control is the level of ADCC activity when the steps of the method are not carried out. In exemplary aspects, the control is the level of ADCC activity when the relative unpaired afucosylated glycan content and/or the relative unpaired high mannose glycan content is not modified (increased or decreased). For example, a suitable control may be the ADCC activity level of the same protein or antibody composition but without the increase in the relative unpaired glycan content (e.g., relative unpaired afucosylated glycan content and/or the relative unpaired high mannose glycan content), or a suitable control may be the ADCC activity level of the same protein or antibody composition but without the decrease in the relative unpaired glycan content (e.g., relative unpaired afucosylated glycan content and/or the relative unpaired high mannose glycan content). In exemplary instances, the control may be the ADCC activity level of the same protein or antibody composition produced under the same cell culture conditions with exception of those conditions that were modified to cause a change in the relative unpaired glycan content. In exemplary aspects, the control may be the ADCC activity level of the same protein or antibody composition produced under a first set of cell culture conditions which lead to an ADCC activity level which is outside of a target range of ADCC activity level. In various aspects, the control may be the ADCC activity level of the same protein or antibody composition produced under a first set of cell culture conditions which lead to a relative unpaired afucosylated glycan content and/or the relative unpaired high mannose glycan content which is/are outside of a target range.

The term “target range” refers to a range of values based on a reference standard or reference product. For instance, the target range of ADCC activity level may be the range of ADCC activity level exhibited by a reference product. Also, for example, the target range of relative unpaired glycan content (e.g., relative unpaired afucosylated glycan content and/or the relative unpaired high mannose glycan content) may be the range of relative unpaired glycan content (e.g., relative unpaired afucosylated glycan content and/or the relative unpaired high mannose glycan content) for a reference product. In various aspects, the target range is a predetermined target range, meaning that the target range was determined or ascertained at a prior timepoint.

### Methods of Modifying Unpaired Glycan Content

In exemplary embodiments, the method of modifying (increasing or decreasing) the ADCC level of an antibody composition comprises modifying (increasing or decreasing) the unpaired afucosylated glycan content of an antibody composition and/or the unpaired high mannose glycan content of an antibody composition. In exemplary aspects, the presently disclosed method of modifying the ADCC level of an antibody composition comprises increasing the unpaired afucosylated glycan content and/or the unpaired high mannose content to increase the level of ADCC activity. In various aspects, the method of modifying the ADCC level of an antibody composition comprises increasing the unpaired afucosylated glycan content and/or the unpaired high mannose content by at least about 1%, at least about 2%, at least about 3%, at least about 4%, at least about 5%, or more. In various aspects, the method of modifying the ADCC level of an antibody composition comprises increasing the unpaired afucosylated glycan content and/or the unpaired high mannose content by more than 5% or more than 10%, e.g., by about 5%, about 6%, about 7%, about 8%, about 9%, about 10%, about 11%, about 12%, about 13%, about 14%, about 15%, about 16%, about 17%, about 18%, about 19%, or about 20%. In various aspects, the method comprises increasing the unpaired afucosylated glycan content and/or the unpaired high mannose content by at least about 25%, at least about 30%, at least about 40%, at least about 50%, at least about 60%, at least about 70%, at least about 80%, at least about 90%, or more.

In exemplary aspects, the presently disclosed method of modifying the ADCC level of an antibody composition comprises decreasing the unpaired afucosylated glycan content to decrease the level of ADCC activity. In various aspects, the method of modifying the ADCC level of an antibody composition comprises decreasing the unpaired afucosylated glycan content and/or the unpaired high mannose content by at least about 1%, at least about 2%, at least about 3%, at least about 4%, at least about 5%, or more. In various aspects, the method of modifying the ADCC level of an antibody composition comprises decreasing the unpaired afucosylated glycan content and/or the unpaired high mannose content by more than 5% or more than 10%, e.g., by about 5%, about 6%, about 7%, about 8%, about 9%, about 10%, about 11%, about 12%, about 13%, about 14%, about 15%, about 16%, about 17%, about 18%, about 19%, or about 20%. In various aspects, the method comprises decreasing the unpaired afucosylated glycan content and/or the unpaired high mannose content by at least about 25%, at least about 30%, at least about 40%, at least about 50%, at least about 60%, at least about 70%, at least about 80%, at least about 90%, or more.

In exemplary aspects, the increase or decrease in the unpaired afucosylated glycan content and/or unpaired high mannose glycan content is/are relative to a “control”. In exemplary aspects, the control is the unpaired glycan content of a control protein or antibody composition produced under the same cell culture conditions with exception of those conditions that lead to increased or decreased unpaired glycan content. In exemplary aspects, the control may be the unpaired glycan content of the same protein or antibody composition produced under a first set of cell culture conditions which lead to an ADCC activity level which is outside of a target range of ADCC activity level. In various aspects, the control may be the unpaired glycan content of the same protein or antibody composition produced under a first set of cell culture conditions which lead to an unpaired afucosylated glycan content and/or the unpaired high mannose glycan content which is/are outside of a target range.

Without being bound to a particular theory, conditions which lead to a modified (increased or decreased) afucosylated glycan content and/or high mannose content may lead to increased or decreased unpaired afucosylated glycan content and/or unpaired high mannose content of an antibody composition. In various instances, the unpaired afucosylated glycan content and/or unpaired high mannose content is increased or decreased by following the teachings of any one of International Patent Application Publication Nos. WO2013/114164, WO2013/114245, WO2013/114167, WO2015128793, or WO2016/089919, WO2018/170099, WO2019/191150, each of which is incorporated herein by reference. In various instances, the unpaired afucosylated glycan content and/or unpaired high mannose content is increased or decreased by selecting a clone that produces antibody or antibody protein product comprising a level of unpaired afucosylated glycan content and/or unpaired high mannose content within a target range.

### Methods of Producing Antibody Compositions

Simple and efficient methods to predict the level of effector function (e.g., ADCC) a particular antibody composition will exhibit based on a given glycoform profile for that antibody composition are described herein. The data provided herein support that the ADCC activity level for the antibody composition may be predicted based on the relative unpaired glycan content of an antibody composition. Without being bound to a particular theory, the unpaired afucosylated glycan content and/or the relative unpaired high mannose glycan content of an antibody composition is predictive of the ADCC activity level of the antibody composition, Such predicted ADCC levels are useful during antibody production, when, it is necessary for the antibody to have an ADCC activity level within a target range. For instance, by monitoring the unpaired afucosylated glycan content and/or the unpaired high mannose glycan content of an antibody composition, it may be predicted whether the antibody composition will exhibit an ADCC activity level within a target range. If a target range of ADCC activity levels for an antibody composition is known, the target range of relative unpaired afucosylated glycan content and/or the relative unpaired high mannose glycan content may be determined. Selection of the antibody composition for continued processing, e.g., downstream processing, may occur when the unpaired afucosylated glycan content and/or the unpaired high mannose glycan, or the ADCC activity level, as calculated based on the unpaired afucosylated glycan content and/or the unpaired high mannose glycan content, is/are within a target range. In exemplary aspects, the target range is based on a target range of ADCC activity levels for a reference antibody and a model which correlates ADCC activity level of the antibody composition to afucosylated glycan content and/or high mannose glycan content of the antibody composition, optionally, a model which correlates ADCC activity level of the antibody composition to unpaired afucosylated glycan content and/or unpaired high mannose glycan content of the antibody composition.

Accordingly, the present disclosure provides methods of producing an antibody composition. In exemplary embodiments, the method comprises (i) determining the relative unpaired afucosylated glycan content and/or the relative unpaired high mannose glycan content of a sample of the antibody composition; and (ii) selecting the antibody composition for downstream processing based on the relative unpaired afucosylated glycan content and/or relative unpaired high mannose glycan content determined in (i). In exemplary embodiments, the method of producing an antibody composition comprises (i) determining the unpaired afucosylated glycan content of an antibody composition and/or the unpaired high mannose glycan content of an antibody composition; (ii) determining the ADCC level of the antibody composition based on the unpaired afucosylated glycan content and/or the unpaired high mannose glycan content determined in (i); and (iii) selecting the antibody composition for downstream processing when the ADCC level of the antibody composition determined in (ii) is within a target ADCC range. In exemplary embodiments, the method of producing an antibody composition comprises (i) determining the unpaired afucosylated glycan content and/or the unpaired high mannose glycan content of a sample of the antibody composition taken from a cell culture comprising glycosylation-competent cells expressing an antibody of the antibody composition; (ii) optionally, modifying the cell culture to modulate the unpaired afucosylated glycan content and/or the unpaired high mannose glycan content and determining the unpaired afucosylated glycan content and/or the unpaired high mannose glycan content of a sample of the antibody composition taken from the modified cell culture; and (iii) selecting the antibody composition for downstream processing based on the unpaired afucosylated glycan content and/or unpaired high mannose glycan content.

Also provided herein are methods of preparing an IgG antibody composition having an ADCC activity level within a target range. In exemplary embodiments, the method comprises determining the relative unpaired glycan content of a sample of the IgG antibody composition in accordance with any one of the presently disclosed methods of determining the relative unpaired glycan content. The relative unpaired AF glycan content and/or relative unpaired HM glycan content of the IgG antibody composition may be compared to a target range of unpaired AF glycan content and/or relative unpaired HM glycan content. For example, the target range may be a reference level of a specification, or of a reference IgG antibody composition. Also, for instance, the target range of unpaired AF glycan content and/or relative unpaired HM glycan content may be a predetermined target range of unpaired AF glycan content and/or relative unpaired HM glycan content which statistically correlates with a predetermined target range of ADCC activity levels of a reference IgG antibody composition.

Methods of producing an antibody composition are also provided by the present disclosure. In exemplary embodiments, the method comprises determining relative unpaired glycan content of the antibody composition is determined in accordance with any one of the presently disclosed methods of determining the relative unpaired glycan content, wherein, when the relative unpaired glycan content is determined as outside a predetermined target range, the method further comprises modifying one or more conditions of the cell culture to obtain a modified cell culture and determining the relative unpaired glycan content. The method in various instance comprises repeating steps of the method until the relative unpaired glycan content is within the predetermined target range.

In various aspects, the sample is taken from a cell culture comprising glycosylation-competent cells expressing an antibody of the antibody composition. Optionally, the method further comprises modifying one or more conditions of the cell culture to modify the relative unpaired afucosylated glycan content and/or the relative unpaired high mannose glycan content of the antibody composition and determining the relative unpaired afucosylated glycan content and/or the relative unpaired high mannose glycan content of a sample of the antibody composition taken from the modified cell culture. In exemplary aspects, the method comprises repeating the modifying until the relative unpaired afucosylated glycan content and/or relative unpaired high mannose glycan content is within a target range. In various aspects, the relative unpaired afucosylated glycan content and/or the relative unpaired high mannose glycan content is/are determined in real time with respect to production of the antibody composition. As used herein “real time” refers to determinations that are made while a production process is ongoing, without interruption to the process. It will be appreciated that production of therapeutic proteins involves living cells and sensitive materials that cannot be put on hold indefinitely while assays and determinations are performed. If numerical examples of interest, a “real time” determination can be a determination that is made within 6 hours, 5 hours, 4 hours, 3 hours, 2 hours, 1 hour, 50 minutes, 40 minutes, 30 minutes, 20 minute, 10 minutes, 5 minutes, 1 minute, 30 seconds, 20 seconds, 10 seconds, 5 seconds, 1 seconds, or 0.1 seconds from the time a measurement is made (while the production process is ongoing). In various instances, the method comprises selecting the antibody composition for downstream processing when the relative unpaired afucosylated glycan content and/or relative unpaired high mannose glycan content is/are in a target range. In various aspects, the relative unpaired afucosylated glycan content and/or relative unpaired high mannose glycan content correlate with the ADCC activity level of the antibody composition. Optionally, the method further comprises determining the ADCC activity level of the antibody composition based on the relative unpaired afucosylated glycan content and/or relative unpaired high mannose glycan content determined in (i). Optionally, the method comprises selecting the antibody composition for downstream processing when the ADCC activity level is in a target range.

In various aspects, the method of producing an antibody composition comprises modifying the ADCC level of an antibody composition according to a method of modifying the ADCC level of the present disclosure.

In various instances, the method of producing an antibody composition comprises determining the relative unpaired glycan content of an antibody composition according to any of the presently disclosed methods of determining the relative unpaired glycan content of an antibody composition.

### Downstream Processing

The relative % unpaired high mannose glycans and/or relative % unpaired afucosylated glycans are determined (e.g., measured) to better inform as to the % antibody-dependent cell-mediated cytotoxicity (ADCC) of the antibody composition. The determining (e.g., measuring) may occur at any stage of manufacture. In particular, measurements may be taken pre- or post-harvest, preceding or during any stage of downstream processing. Example downstream processing includes any chromatography unit operation, including capture chromatography, intermediate chromatography, and/or polish chromatography unit operations; virus inactivation and neutralization; virus filtration; and/or final formulation. The relative % unpaired high mannose glycans, and/or relative % unpaired afucosylated glycans in various aspects is determined (e.g., measured) in real-time, near real-time, and/or after the fact, Monitoring and measurements can be done using known techniques and commercially available equipment.

In various aspects of the present disclosure, the determining (e.g., measuring) the % unpaired high mannose glycans and/or % unpaired afucosylated glycans is carried out before a harvest. As used herein the term “harvest” refers to cell culture media containing the recombinant protein of interest being collected and separated at least from the cells of the cell culture. The harvest can be performed continuously. The harvest in some aspects is performed using centrifugation and can further comprise precipitation, filtration, and the like. In various aspects, the determining is carried out before harvest. In various aspects, the determining is carried out before chromatography, optionally, Protein A chromatography. In some aspects, the determining (e.g., measuring) the relative % unpaired high mannose glycans and/or relative % unpaired afucosylated glycans is carried out at least 3 days, at least 4 days, or at least 5 days before harvest. Optionally, determining (e.g., measuring) the relative % unpaired high mannose glycans and/or relative % unpaired afucosylated glycans is carried out in real-time with regard to antibody production.

In various aspects of the present disclosure, determining (e.g., measuring) the relative % unpaired high mannose glycans, and/or relative % unpaired afucosylated glycans is carried out after a harvest. In various aspects, the determining is carried out after chromatography, optionally, Protein A chromatography. In various aspects, the determining is carried out after harvest and after chromatography, e.g., a Protein A chromatography.

With regard to the presently disclosed methods, the antibody composition in various aspects is selected or chosen for further processing, e.g., for downstream processing, and the selection is based on a particular parameter, e.g., % ADCC, relative % unpaired high mannose glycans, and/or relative % unpaired afucosylated glycans. In various instances, the presently disclosed methods comprise using the antibody composition in further processing, e.g., downstream processing, based on a particular parameter, e.g., based on the % ADCC, relative % unpaired high mannose glycans, and/or relative % unpaired afucosylated glycans. In various instances, the presently disclosed methods comprise carrying out further processing, e.g., downstream processing, with the antibody composition, based on a particular parameter, e.g., based on the % ADCC, relative % unpaired high mannose glycans, and/or relative % unpaired afucosylated glycans.

In exemplary instances the downstream processing comprises or consists of any processing which occurs after (or downstream of) the processing at which the relative % unpaired high mannose glycans, and/or relative % unpaired afucosylated glycans are determined (e.g., measured). For example, if the relative % unpaired high mannose glycans, and/or relative % unpaired afucosylated glycans were determined (e.g., measured) at harvest, then the downstream processing is any processing which occurs after (or downstream of) the harvest, which in various aspects comprise(s): dilution, filling, filtration, formulation, chromatography, viral filtration, viral inactivation, or a combination thereof. Also, for example, if the relative % unpaired high mannose glycans, and/or relative % unpaired afucosylated glycans were determined (e.g., measured) after chromatography, e.g., Protein A chromatography, then the downstream processing comprises or consists of any processing which occurs after (or downstream of) the chromatography, and the downstream processing in various aspects comprise(s): a dilution, a filling, a filtration, a formulation, further chromatography, a viral filtration, a viral inactivation, or a combination thereof. In exemplary instances the further chromatography is an ion exchange chromatography (e.g., cation exchange chromatography or anion exchange chromatography).

Stages/types of chromatography used during downstream processing include capture or affinity chromatography which is used to separate the recombinant product from other proteins, aggregates, DNA, viruses and other such impurities, in exemplary instances, initial chromatography is carried out with Protein A (e.g., Protein A attached to a resin). Intermediate and polish chromatography in various aspects further purify the recombinant protein, removing bulk contaminants, adventitious viruses, trace impurities, aggregates, isoforms, etc. The chromatography can either be performed in bind and elute mode, where the recombinant protein of interest is bound to the chromatography medium and the impurities flow through, or in flow-through mode, where the impurities are bound and the recombinant protein flows through. Examples of such chromatography methods include ion exchange chromatography (IEX), such as anion exchange chromatography (AEX) and cation exchange chromatography (CEX); hydrophobic interaction chromatography (HIC); mixed modal or multimodal chromatography (MM), hydroxyapatite chromatography (HA); reverse phase chromatography and gel filtration.

In various aspects, the downstream processing comprises viral inactivation. Enveloped viruses have a capsid enclosed by a lipoprotein membrane or “envelope” and are therefore susceptible to inactivation. The virus inactivation in various instances includes heat inactivation/pasteurization, pH inactivation, UV and gamma ray irradiation, use of high intensity broad spectrum white light, addition of chemical inactivating agents, surfactants, and solvent/detergent treatments.

In various aspects, the downstream processing comprises virus filtration. In various aspects, the virus filtration comprises removing non-enveloped viruses. In various aspects, the virus filtration comprises the use of micro- or nano-filters.

In various aspects, the downstream processing comprises formulation, which may be performed in one or more steps. Following completion of the chromatography, the purified recombinant proteins are in various aspects buffer exchanged into a formulation buffer. In exemplary aspects, the buffer exchange is performed using ultrafiltration and diafiltration (UF/DF). In exemplary aspects, the recombinant protein is buffer exchanged into a desired formulation buffer using diafiltration and concentrated to a desired final formulation concentration using ultrafiltration. Additional stability-enhancing excipients in various aspects are added following a UF/DF formulation.

### Additional Steps

The methods disclosed herein, in various aspects, comprise additional steps. For example, in some aspects, the methods comprise one or more upstream steps or downstream steps involved in producing, purifying, and formulating an antibody composition. Optionally, the downstream steps are any one of those downstream processing steps described herein or known in the art. See, e.g., Downstream Processing. In exemplary embodiments, the method comprises steps for generating host cells that express a recombinant glycosylated protein (e.g., antibody). The host cells, in some aspects, are prokaryotic host cells, e.g., E. coli or Bacillus subtills, or the host cells, in some aspects, are eukaryotic host cells, e.g., yeast cells, filamentous fungi cells, protozoa cells, insect cells, or mammalian cells (e.g., CHO cells). Such host cells are described in the art. See, e.g., Kunert et al., Appl. Microbiol Biotechnol. 100: 3451-61 (2016) and herein under “Cells.” For example, the methods comprise, in some instances, introducing into host cells a vector comprising a nucleic acid comprising a nucleotide sequence encoding the recombinant glycosylated protein, or a polypeptide chain thereof.

In exemplary aspects, the methods comprise maintaining cells, e.g., glycosylation-competent cells in a cell culture. Accordingly, the methods may comprise carrying out any one or more steps described herein in Maintaining Cells in A Cell Culture.

In exemplary embodiments, the methods disclosed herein comprise steps for isolating and/or purifying the antibodies from the culture. In exemplary aspects, the method comprises one or more chromatography steps including, but not limited to, e.g., affinity chromatography (e.g., protein A affinity chromatography), ion exchange chromatography, and/or hydrophobic interaction chromatography. In exemplary aspects, the method comprises steps for producing crystalline biomolecules from a solution comprising the recombinant glycosylated proteins.

The methods of the disclosure, in various aspects, comprise one or more steps for preparing a composition, including, in some aspects, a pharmaceutical composition, comprising the purified recombinant glycosylated protein. Such compositions are discussed herein.

### Maintaining Cells in a Cell Culture

With regard to the methods of producing an antibody composition of the present disclosure, the antibody composition may be produced by maintaining cells in a cell culture. The cell culture may be maintained according to any set of conditions suitable for production of a recombinant glycosylated protein. For example, in some aspects, the cell culture is maintained at a particular pH, temperature, cell density, culture volume, dissolved oxygen level, pressure, osmolality, and the like. In exemplary aspects, the cell culture prior to inoculation is shaken (e.g., at 70 rpm) at 5% CO₂ under standard humidified conditions in a CO₂ incubator. In exemplary aspects, the cell culture is inoculated with a seeding density of about 10⁶ cells/mL in 1.5 L medium.

In exemplary aspects, the methods of the disclosure comprise maintaining the glycosylation-competent cells in a cell culture medium at a pH of about 6.85 to about 7.05, e.g., in various aspects, about 6.85, about 6.86, about 6.87, about 6.88, about 6.89, about 6,90, about 6.91, about 6.92, about 6.93, about 694, about 6.95, about 696, about 6.97, about 698, about 6.99, about 7.00, about 7.01, about 7.02, about 7.03, about 7.04, or about 7.05.

In exemplary aspects, the methods comprise maintaining the cell culture at a temperature between 30° C. and 40° C. In exemplary embodiments, the temperature is between about 32° C. to about 38° C. or between about 35° C. to about 38° C.

In exemplary aspects, the methods comprise maintaining the osmolality between about 200 mOsm/kg to about 500 mOsm/kg, in exemplary aspects, the method comprises maintaining the osmolality between about 225 mOsm/kg to about 400 mOsm/kg or about 225 mOsm/kg to about 375 mOsm/kg. In exemplary aspects, the method comprises maintaining the osmolality between about 225 mOsm/kg to about 350 mOsm/kg. In various aspects, osmolality (mOsm/kg) is maintained at about 200, 225, about 250, about 275, about 300, about 325, about 350, about 375, about 400, about 425, about 450, about 475, or about 500.

In exemplary aspects, the methods comprise maintaining dissolved the oxygen (DO) level of the cell culture at about 20% to about 60% oxygen saturation during the initial cell culture period. In exemplary instances, the method comprises maintaining DO level of the cell culture at about 30% to about 50% (e.g., about 35% to about 45%) oxygen saturation during the initial cell culture period. In exemplary instances, the method comprises maintaining DO level of the cell culture at about 20%, about 25%, about 30%, about 35%, about 40%, about 45%, about 50%, about 55%, or about 60% oxygen saturation during the initial cell culture period. In exemplary aspects, the DO level is about 35 mm Hg to about 85 mmHg or about 40 mm Hg to about 80 mmHg or about 45 mm Hg to about 75 mm Hg.

The cell culture is maintained in any one or more culture medium. In exemplary aspects, the cell culture is maintained in a medium suitable for cell growth and/or is provided with one or more feeding media according to any suitable feeding schedule. In exemplary aspects, the method comprises maintaining the cell culture in a medium comprising glucose, fucose, lactate, ammonia, glutamine, and/or glutamate. In exemplary aspects, the method comprises maintaining the cell culture in a medium comprising manganese at a concentration less than or about 1 μM during the initial cell culture period. In exemplary aspects, the method comprises maintaining the cell culture in a medium comprising about 0.25 μM to about a 1 μM manganese. In exemplary aspects, the method comprises maintaining the cell culture in a medium comprising negligible amounts of manganese. In exemplary aspects, the method comprises maintaining the cell culture in a medium comprising copper at a concentration less than or about 50 ppb during the initial cell culture period. In exemplary aspects, the method comprises maintaining the cell culture in a medium comprising copper at a concentration less than or about 40 ppb during the initial cell culture period. In exemplary aspects, the method comprises maintaining the cell culture in a medium comprising copper at a concentration less than or about 30 ppb during the initial cell culture period. In exemplary aspects, the method comprises maintaining the cell culture in a medium comprising copper at a concentration less than or about 20 ppb during the initial cell culture period. In exemplary aspects, the medium comprises copper at a concentration greater than or about 5 ppb or greater than or about 10 ppb. In exemplary aspects, the cell culture medium comprises mannose. In exemplary aspects, the cell culture medium does not comprise mannose.

In exemplary embodiments, the type of cell culture is a fed-batch culture or a continuous perfusion culture. However, the methods of the disclosure are advantageously not limited to any particular type of cell culture.

The cells maintained in cell culture may be glycosylation-competent cells. In exemplary aspects, the glycosylation-competent cells are eukaryotic cells, including, but not limited to, yeast cells, filamentous fungi cells, protozoa cells, algae cells, insect cells, or mammalian cells. Such host cells are described in the art. See, e.g., Kunert et al., Appl. Microbiol Biotechnol. 100: 3451-61 (2016). In exemplary aspects, the eukaryotic cells are mammalian cells. In exemplary aspects, the mammalian cells are non-human mammalian cells. In some aspects, the cells are Chinese Hamster Ovary (CHO) cells and derivatives thereof (e.g., CHO-K1, CHO pro-3), mouse myeloma cells (e.g., NSO, GS-NSO, Sp2/0), cells engineered to be deficient in dihydrofolatereductase (DHFR) activity (e.g., DLKX-X11, 0G44), human embryonic kidney 293 (HEK293) cells or derivatives thereof (e.g., HEK293T, HEK293-EBNA), green African monkey kidney cells (e.g., COS cells, VERO cells), human cervical cancer cells (e.g., HeLa), human bone osteosarcoma epithelial cells U2-OS, adenocarcinomic human alveolar basal epithelial cells A549, human fibrosarcoma cells HT1080, mouse brain tumor cells CAD, embryonic carcinoma cells P19, mouse embryo fibroblast cells NIH 3T3, mouse fibroblast cells L929, mouse neuroblastoma cells N2a, human breast cancer cells MCF-7, retinoblastoma cells Y79, human retinoblastoma cells SO-Rb50, human liver cancer cells Hep G2, mouse B myeloma cells J558L, or baby hamster kidney (BHK) cells (Gaillet et al. 2007; Khan, Adv Pharm Bull 3(2): 257-263 (2013)).

Cells that are not glycosylation-competent can also be transformed into glycosylation-competent cells, e.g. by transfecting them with genes encoding relevant enzymes necessary for glycosylation. Exemplary enzymes include but are not limited to oligosaccharyltransferases, glycosidases, glucosidase I, glucosidase II, calnexin/calreticulin, glycosyltransferases, mannosidases, GlcNAc transferases, galactosyltransferases, and sialyltransferases.

In exemplary embodiments, the glycosylation-competent cells are not genetically modified to alter the activity of an enzyme of the de novo pathway or the salvage pathway. These two pathways of fucose metabolism are shown in FIG. 2. In exemplary embodiments, the glycosylation-competent cells are not genetically modified to alter the activity of any one or more of: a fucosyl-transferase (FUT, e.g., FUT1, FUT2, FUT3, FUT4, FUT5, FUT6, FUT7, FUT8, FUT9), a fucose kinase, a GDP-fucose pyrophosphorylase, GDP-D-mannose-4,6-dehydratase (GMD), and GDP-keto-6-deoxymannose-3,5-epimerase, 4-reductase (FX). In exemplary embodiments, the glycosylation-competent cells are not genetically modified to knock-out a gene encoding FX.

In exemplary embodiments, the glycosylation-competent cells are not genetically modified to alter the activity β(1,4)-N-acetylglucosaminyltransferase III (GNTIII) or GDP-6-deoxy-D-lyxo-4-hexulose reductase (RMD). In exemplary aspects, the glycosylation-competent cells are not genetically modified to overexpress GNTIII or RMD.

### Antibodies and Fragments Thereof

As used herein, the term “antibody” refers to a protein having a conventional immunoglobulin format, comprising heavy and light chains, and comprising variable and constant regions. For example, an antibody may be an IgG which is a “Y-shaped” structure of two identical pairs of polypeptide chains, each pair having one “light” (typically having a molecular weight of about 25 kDa) and one “heavy” chain (typically having a molecular weight of about 50-70 kDa). An antibody has a variable region and a constant region. In IgG formats, the variable region is generally about 100-110 or more amino acids, comprises three complementarity determining regions (CDRs), is primarily responsible for antigen recognition, and substantially varies among other antibodies that bind to different antigens, See, e.g., Janeway et al., “Structure of the Antibody Molecule and the Immunoglobulin Genes”, Immunobiology: The Immune System in Health and Disease, 4th ed. Elsevier Science Ltd./Garland Publishing, (1999).

Briefly, in an antibody scaffold, the CDRs are embedded within a framework in the heavy and light chain variable region where they constitute the regions largely responsible for antigen binding and recognition. A variable region comprises at least three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342: 877-883), within a framework region (designated framework regions 1-4, FR1, FR2, FR3, and FR4, by Kabat et al., 1991; see also Chothia and Lesk, 1987, supra).

Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and Ig4. IgM has subclasses, including, but not limited to, IgM1 and IgM2. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. In various aspects, the antibody is an IgG, including any one of IgG1, IgG2, IgG3 or IgG4. In various instances, the antibody is an IgG1.

In various aspects, the antibody can be a monoclonal antibody or a polyclonal antibody. In exemplary instances, the antibody is a mammalian antibody, e.g., a mouse antibody, rat antibody, rabbit antibody, goat antibody, horse antibody, chicken antibody, hamster antibody, pig antibody, human antibody, and the like. In certain aspects, the recombinant glycosylated protein is a monoclonal human antibody.

In various aspects, the antibody is a chimeric antibody or a humanized antibody. The term “chimeric antibody” is used herein to refer to an antibody containing constant domains from one species and the variable domains from a second, or more generally, containing stretches of amino acid sequence from at least two species. The term “humanized” when used in relation to antibodies refers to antibodies having at least CDR regions from a non-human source which are engineered to have a structure and immunological function more similar to true human antibodies than the original source antibodies. For example, humanizing can involve grafting CDR from a non-human antibody, such as a mouse antibody, into a human antibody. Humanizing also can involve select amino acid substitutions to make a non-human sequence look more like a human sequence.

An antibody, in various aspects, is cleaved into fragments by enzymes, such as, e.g., papain and pepsin. Papain cleaves an antibody to produce two Fab fragments and a single Fc fragment. Pepsin cleaves an antibody to produce a F(ab′)2 fragment and a pFc′ fragment. As described herein, the methods of determining unpaired glycan content comprises a two-enzyme digestion, one of which is used to produce a mixture of Fab fragments and Fc fragments.

Advantageously, the methods are not limited to the antigen-specificity of the antibody. Accordingly, the antibody has any binding specificity for virtually any antigen. In exemplary aspects, the antibody binds to a hormone, growth factor, cytokine, a cell-surface receptor, or any ligand thereof. In exemplary aspects, the antibody binds to a protein expressed on the cell surface of an immune cell. In exemplary aspects, the antibody binds to a cluster of differentiation molecule selected from the group consisting of: CD1a, CD1b, CD1c, CD1d, CD2, CD3, CD4, CDS, CD6, CD7, CD8, CD9, CD10, CD11A, CD11B, CD11C, CDw12, CD13, CD14, CD15, CD15s, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L, CD62P, CD63, CD64, CD65, CD66a, CD66b, CD66c, CD66d, CD66e, CD66f, CD68, CD69, CD70, CD71, CD72, CD73, CD74, CD75, CD76, CD79a, CD79@, CD80, CD81, CD82, CD83, CDw84, CD85, CD86, CD87, CD88, CD89, CD90, CD91, CDw92, CD93, CD94, CD95, CD96, CD97, CD98, CD99, CD100, CD101, CD102, CD103, CD104, CD105, CD106, CD107a, CD107b, CDw108, CD109, CD114, CD 115, CD116, CD117, CD118, CD119, CD120a, CD120b, CD121a, CDw121b, CD122, CD123, CD124, CD125, CD126, CD127, CDw128, CD129, CD130, CDw131, CD132, CD134, CD135, CDw136, CDw137, CD138, CD139, CD140a, CD140b, CD141, CD142, CD143, CD144, CD145, CD146, CD147, CD148, CD150, CD151, CD152, CD153, CD154, CD155, CD156, CD157, CD158a, CD158b, CD161, CD162, CD163, CD164, CD165, CD166, and CD182.

In exemplary aspects, the antibody is one of those described in U.S. Pat. No. 7,947,809 and U.S. Patent Application Publication No. 20090041784 (glucagon receptor), U.S. Pat. Nos. 7,939,070, 7,833,527, 7,767,206, and 7,786,284 (IL-17 receptor A), U.S. Pat. Nos. 7,872,106 and 7,592,429 (Sclerostin), U.S. Pat. Nos. 7,871,611, 7,815,907, 7,037,498, 7,700,742, and U.S. Patent Application Publication No. 20100255538 (IGF-1 receptor), U.S. Pat. No. 7,868,140 (B7RP1), U.S. Pat. No. 7,807,159 and U.S. Patent Application Publication No. 20110091455 (myostatin), U.S. Pat. Nos. 7,736,644, 7,628,986, 7,524,496, and U.S. Patent Application Publication No. 20100111979 (deletion mutants of epidermal growth factor receptor), U.S. Pat. No. 7,728,110 (SARS coronavirus), U.S. Pat. No. 7,718,776 and U.S. Patent Application Publication No. 20100209435 (OPGL), U.S. Pat. Nos. 7,658,924 and 7,521,053 (Angiopoietin-2), U.S. Pat. Nos. 7,601,818, 7,795,413, U.S. Patent Application Publication No. 20090155274, U.S. Patent Application Publication No. 20110040076 (NGF), U.S. Pat. No. 7,579,186 (TGF-β type II receptor), U.S. Pat. No. 7,541,438 (connective tissue growth factor), U.S. Pat. No. 7,438,910 (IL1-R1), U.S. Pat. No. 7,423,128 (properdin), U.S. Pat. Nos. 7,411,057, 7,824,679, 7,109,003, 6,682,736, 7,132,281, and 7,807,797 (CTLA-4), U.S. Pat. Nos. 7,084,257, 7,790,859, 7,335,743, 7,084,257, and U.S. Patent Application Publication No. 20110045537 (interferon-gamma), U.S. Pat. No. 7,932,372 (MAdCAM), U.S. Pat. No. 7,906,625, U.S. Patent Application Publication No. 20080292639, and U.S. Patent Application Publication No. 20110044986 (amyloid), U.S. Pat. Nos. 7,815,907 and 7,700,742 (insulin-like growth factor I), U.S. Pat. Nos. 7,566,772 and 7,964,193 (interleukin-13), U.S. Pat. Nos. 7,563,442, 7,288,251, 7,338,660, 7,626,012, 7,618,633, and U.S. Patent Application Publication No. 20300098694 (CD40), U.S. Pat. No. 7,498,420 (c-Met), U.S. Pat. Nos. 7,326,414, 7,592,430, and 7,728,113 (M-CSF), U.S. Pat. Nos. 6,924,360, 7,067,131, and 7,090,844 (MUC18), U.S. Pat. Nos. 6,235,883, 7,807,798, and U.S. Patent Application Publication No. 20100305307 (epidermal growth factor receptor), U.S. Pat. Nos. 6,716,587, 7,872,113, 7,465,450, 7,186,809, 7,317,090, and 7,638,606 (interleukin-4 receptor), U.S. Patent Application Publication No. 20110135657 (BETA-KLOTHO), U.S. Pat. Nos. 7,887,799 and 7,879,323 (fibroblast growth factor-like polypeptides), U.S. Pat. No. 7,867,494 (IgE), U.S. Patent Application Publication No. 20100254975 (ALPHA-4 BETA-7), U.S. Patent Application Publication No. 20100197005 and U.S. Pat. No. 7,537,762 (ACTIVIN RECEPTOR-LIKE KINASE-1), U.S. Pat. No. 7,585,500 and U.S. Patent Application Publication No. 20100047253 (IL-13), U.S. Patent Application Publication No. 20090263383 and U.S. Pat. No. 7,449,555 (CD148), U.S. Patent Application Publication No. 20090234106 (ACTIVIN A), U.S. Patent Application Publication No. 20090226447 (angiopoietin-1 and angiopoietin-2), U.S. Patent Application Publication No. 20090191212 (Angiopoietin-2), U.S. Patent Application Publication No. 20090155164 (C-FMS), U.S. Pat. No. 7,537,762 (activin receptor-like kinase-1), U.S. Pat. No. 7,371,381 (galanin), U.S. Patent Application Publication No. 20070196376 (INSULIN-LIKE GROWTH FACTORS), U.S. Pat. Nos. 7,267,960 and 7,741,115 (LDCAM), U.S. Pat. No. 7,265,212 (CD45RB), U.S. Pat. No. 7,709,611, U.S. Patent Application Publication No. 20060127393 and U.S. Patent Application Publication No. 20100040619 (DKK1), U.S. Pat. No. 7,807,795, U.S. Patent Application Publication No. 20030103978 and U.S. Pat. No. 7,923,008 (osteoprotegerin), U.S. Patent Application Publication No. 20090208489 (OV064), U.S. Patent Application Publication No. 20080286284 (PSMA), U.S. Pat. No. 7,888,482, U.S. Patent Application Publication No. 20110165171, and U.S. Patent Application Publication No. 20110059063 (PAR2), U.S. Patent Application Publication No. 20110150888 (HEPCIDIN), U.S. Pat. No. 7,939,640 (B7L-1), U.S. Pat. No. 7,915,391 (c-Kit), U.S. Pat. Nos. 7,807,796, 7,193,058, and U.S. Pat. No. 7,427,669 (ULBP), U.S. Pat. Nos. 7,786,271, 7,304,144, and U.S. Patent Application Publication No. 20090238823 (TSLP), U.S. Pat. No. 7,767,793 (SIGIRR), U.S. Pat. No. 7,705,130 (HER-3), U.S. Pat. No. 7,704,501 (ataxin-1-like polypeptide), U.S. Pat. Nos. 7,695,948 and 7,199,224 (TNF-α converting enzyme), U.S. Patent Application Publication No. 20090234106 (ACTIVIN A), U.S. Patent Application Publication No. 20090214559 and U.S. Pat. No. 7,438,910 (IL1-R1), U.S. Pat. No. 7,579,186 (TG-β type II receptor), U.S. Pat. No. 7,569,387 (TNF receptor-like molecules), U.S. Pat. No. 7,541,438, (connective tissue growth factor), U.S. Pat. No. 7,521,048 (TRAIL receptor-2), U.S. Pat. Nos. 6,319,499, 7,081,523, and U.S. Patent Application Publication No. 20080182976 (erythropoietin receptor), U.S. Patent Application Publication No. 20080166352 and U.S. Pat. No. 7,435,796 (B7RP1), U.S. Pat. No. 7,423,128 (properdin), U.S. Pat. Nos. 7,422,742 and 7,141,653 (interleukin-5), U.S. Pat. Nos. 6,740,522 and 7,411,050 (RANKL), U.S. Pat. No. 7,378,091 (carbonic anhydrase IX (CA IX) tumor antigen), U.S. Pat. Nos. 7,318,925 and 7,288,253 (parathyroid hormone), U.S. Pat. No. 7,285,269 (TNF), U.S. Pat. Nos. 6,692,740 and 7,270,817 (ACPL), U.S. Pat. No. 7,202,343 (monocyte chemo-attractant protein-1), U.S. Pat. No. 7,144,731 (SCF), U.S. Pat. Nos. 6,355,779 and 7,138,500 (4-1BB). U.S. Pat. No. 7,135,174 (PDGFD), U.S. Pat. Nos. 6,630,143 and 7,045,128 (Flt-3 ligand), U.S. Pat. No. 6,849,450 (metalloproteinase inhibitor), U.S. Pat. No. 6,596,852 (LERK-5), U.S. Pat. No. 6,232,447 (LERK-6), U.S. Pat. No. 6,500,429 (brain-derived neurotrophic factor), U.S. Pat. No. 6,184,359 (epithelium-derived T-cell factor), U.S. Pat. No. 6,143,874 (neurotrophic factor NNT-1), U.S. Patent Application Publication No. 201-10027287 (PROPROTEIN CONVERTASE SUBTILISIN KEXIN TYPE 9 (PCSK9)), U.S. Patent Application Publication No. 20110014201 (IL-18 RECEPTOR), and U.S. Patent Application Publication No. 20090155164 (C-FMS). The above patents and published patent applications are incorporated herein by reference in their entirety for purposes of their disclosure of variable domain polypeptides, variable domain encoding nucleic acids, host cells, vectors, methods of making polypeptides encoding said variable domains, pharmaceutical compositions, and methods of treating diseases associated with the respective target of the variable domain-containing antigen binding protein or antibody.

In exemplary embodiments, the antibody is one of Muromonab-CD3 (product marketed with the brand name Orthoclone Okt3®), Abciximab (product marketed with the brand name Reopro®), Rituximab (product marketed with the brand name MabThera®, Rituxan®), Basiliximab (product marketed with the brand name Simulect®), Daclizumab (product marketed with the brand name Zenapax®), Palivizumab (product marketed with the brand name Synagis®), Infliximab (product marketed with the brand name Remicade®), Trastuzumab (product marketed with the brand name Herceptin®), Alemtuzumab (product marketed with the brand name MabCampath®, Campath-1H®), Adalimumab (product marketed with the brand name Humira®), Tositumomab-I131 (product marketed with the brand name Bexxar®), Efalizumab (product marketed with the brand name Raptiva®), Cetuximab (product marketed with the brand name Erbitux®), Ibritumomab tiuxetan (product marketed with the brand name Zevalin®), Omalizumab (product marketed with the brand name Xolair®), ocrelizumab (product marketed with the brand name Ocrevus®), Bevacizumab (product marketed with the brand name Avastin®), Natalizumab (product marketed with the brand name Tysabri®), Ranibizumab (product marketed with the brand name Lucentis®), Panitumumab (product marketed with the brand name Vectibix®), Eculizumab (product marketed with the brand name Soliris®), Certolizumab pegol (product marketed with the brand name Cimzia®), Golimumab (product marketed with the brand name Simponi®), Canakinumab (product marketed with the brand name Ilaris®), Catumaxomab (product marketed with the brand name Removab®), Ustekinumab (product marketed with the brand name Stelara®), Tocilizumab (product marketed with the brand name RoActemra®, Actemra®), Ofatumumab (product marketed with the brand name Arzerra®), Denosumab (product marketed with the brand name Prolia®), Belimumab (product marketed with the brand name Benlysta®), Raxibacumab, ipilimumab (product marketed with the brand name Yervoy®), and Pertuzumab (product marketed with the brand name Perjeta antibodies, such as daclizumab.

In exemplary aspects, the antibody binds to a tumor associated antigen and is an anti-cancer antibody. Examples of suitable anti-cancer antibodies include, but are not limited to, anti-BAFF antibodies such as belimumab; anti-CD20 antibodies such as rituximab; anti-CD22 antibodies such as epratuzumab; anti-CD25 antibodies such as daclizumab; anti-CD30 antibodies such as iratumumab, anti-CD33 antibodies such as gemtuzumab, anti-CD52 antibodies such as alemtuzumab; anti-CD152 antibodies such as ipilimumab; anti-EGFR antibodies such as cetuximab; anti-HER2 antibodies such as trastuzumab and pertuzumab; anti-IL6 antibodies, such as siltuximab; and anti-VEGF antibodies such as bevacizumab; anti-IL6 receptor antibodies such as tocilizumab.

### Antibody Compositions

The presently disclosed methods relate to antibody compositions. In various aspects, the composition comprises only one type of antibody. In various instances, the composition comprises antibodies wherein each antibody of the composition comprises the same or essentially the amino acid sequence. In various aspects, the composition comprises antibodies wherein each antibody of the composition comprises an amino acid sequence which is at least 90% identical to the amino acid sequences of all other antibodies of the composition. In various aspects, the composition comprises antibodies wherein each antibody of the composition comprises an amino acid sequence which is at least 95%, at least 96%, at least 97%, at least 98%, or at least 99% identical to the amino acid sequences of all other antibodies of the composition. In various aspects, the composition comprises antibodies wherein each antibody of the composition comprises an amino acid sequence which is the same or essentially the same (e.g., at least 90% or at least 95%, at least 96%, at least 97%, at least 98%, or at least 99% identical to the amino acid sequences of all other antibodies of the composition) but the glycan pair classifications of the antibodies of the composition may differ from each other.

In various aspects, the composition comprises only one type of antibody. In various instances, the composition comprises antibodies wherein each antibody of the antibody composition comprises the same or essentially the amino acid sequence. In various aspects, the antibody composition comprises antibodies wherein each antibody of the antibody composition comprises an amino acid sequence which is at least 90% identical to the amino acid sequences of all other antibodies of the antibody composition. In various aspects, the antibody composition comprises antibodies wherein each antibody of the antibody composition comprises an amino acid sequence which is at least 95%, at least 96%, at least 97%, at least 98%, or at least 99% identical to the amino acid sequences of all other antibodies of the antibody composition. In various aspects, the antibody composition comprises antibodies wherein each antibody of the antibody composition comprises an amino acid sequence which is the same or essentially the same (e.g., at least 90% or at least 95%, at least 96%, at least 97%, at least 98%, or at least 99% identical to the amino acid sequences of all other antibodies of the antibody composition) but the glycan pair classifications of the antibodies of the antibody composition may differ from each other. In exemplary aspects, the antibody composition comprises a heterogeneous mixture of antibodies having different glycan pair classifications. In various instances, the antibody composition may be characterized in terms of its unpaired AF glycan content, unpaired HM glycan content, paired AF glycan content, and/or its paired HM glycan content. In various instances, the antibody composition may be characterized in terms of its relative abundance of unpaired AF glycans and/or relative abundance of unpaired HM glycans.

The following examples are given merely to illustrate the present invention and not in any way to limit its scope.

### EXAMPLES

### Example 1

This example describes an exemplary simplified method of determining paired glycan content and unpaired glycan content for an antibody composition.

In a previous study, the paired afucosylated and high mannose glycan content and unpaired afucosylated and high mannose glycan content were determined for two different antibody compositions, and statistical relationships correlating unpaired glycan content of the antibody composition with ADCC activity and FcγRIIIa binding were established. In this previous study, the method used to measure the levels of unpaired or paired glycopairs comprised three steps: an antibody cleavage step, a chromatographic separation step, and a mass spectrometry (MS)-based detection step. Briefly, samples of an antibody composition comprising Antibody A or Antibody B were treated with the IgdE (FabALACTiCA® or FL from Genovis Inc., Cambridge, MA) enzyme which cleaves the heavy chain of antibodies above the hinge to create Fab and Fc antibody fragments. The FL enzyme cleaves between the second and third amino acid of the IgG1 heavy chain sequence KTHTCPP (SEQ ID NO: 1). The resulting Fab fragments and glycosylated Fc fragments were subsequently separated and characterized by hydrophilic interaction liquid chromatography (HILIC) with water/organic with ion-pairing reagent as solvents and MS-based detection. The HILIC separation was carried out on a Waters Acquity UPLC Glycoprotein Amide column over a course of 95 min with mobile phase composition 20:80 (v/v) of water and acetonitrile with 0.1% TFA. At 1 min, a linear gradient of 0.44 mi/min was applied for the next 74 min for separating glycopairs based on their interaction with the stationary phase. MS detection was carried out using an Agilent 6545XT QToF MS with an Agilent Jet Stream (AJS) electrospray ionization source (ESI) with settings of: capillary voltage—4500 V; drying gas—11 mL/min; nebulizer pressure—25 psi and gas temperature—340° C. using a mass range of 1000-3000 m/z. Deconvolution was performed using Bioconfirm B.09.00 (Agilent) using S/N of 30 and output mass range set to 40-60 kDa.

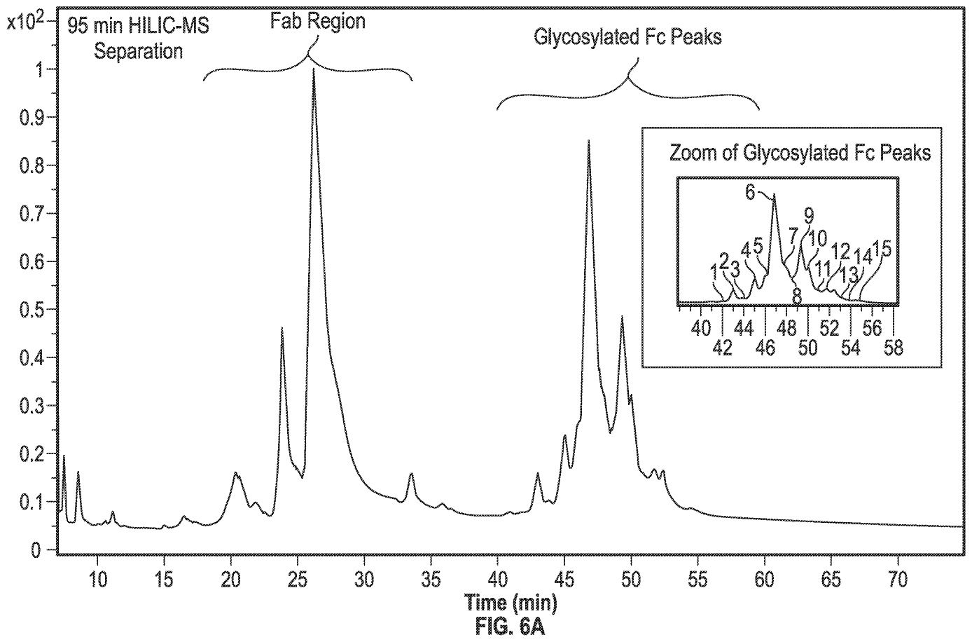

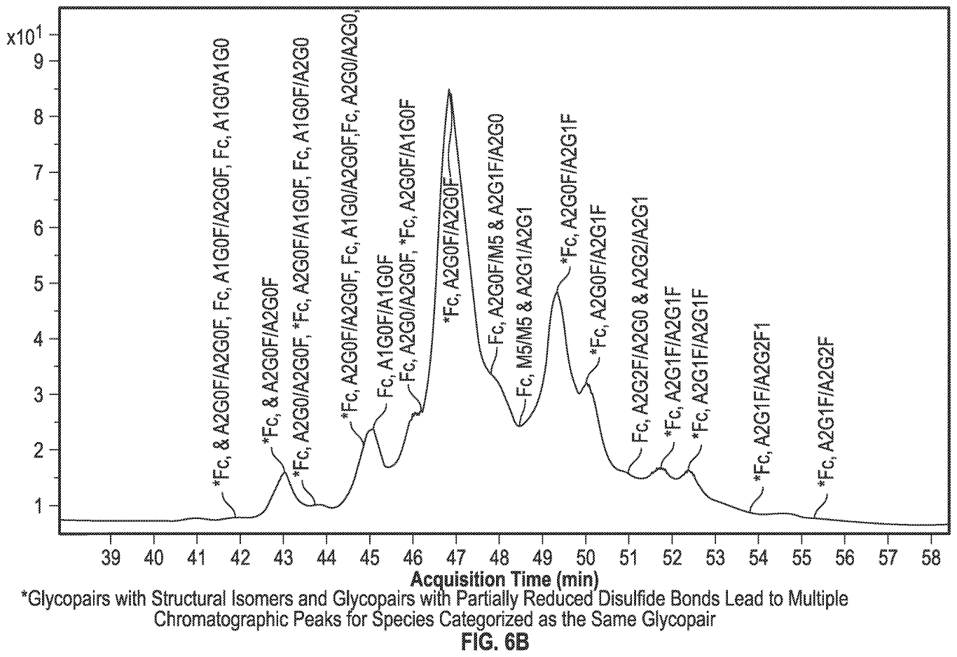

Though the above method successfully measured the content of unpaired and paired afucosylated and high mannose glycans to establish statistically significant correlations with biological functions, a subsequent study was conducted to improve the efficiency of the method for determining the levels of unpaired and paired afucosylated and high mannose glycans. Glycan pairing analysis is complicated due to the high degree of heterogeneity of the glycopair population, and the HILIC separation aims to reduce the complexity through the introduction of a high-resolution separation prior to MS analysis. The glycopairs are separated into a glycopairing map of approximately 15 chromatographic peaks (FIG. 6A). However, the HILIC-MS glycan pairing analysis remains complicated for several reasons. First, the paired fucosylated (Fc, A2G0F/A2G0F, Fc, A2G0F/A2G1F and Fc, A2G1F/A2G1F) glycopairs, which do not significantly impact ADCC, are the dominant species in the analysis, eluting across the entire chromatographic region of the glycosylated Fc fragment chromatogram (FIG. 6A-6B). Second, many of the important chromatographic peaks are poorly resolved: the key afucosylated and high mannose glycopairs co-elute with and/or poorly separate from the dominant paired fucosylated glycopairs. As a result, it is difficult to establish the correct window to extract the mass spectra for each chromatographic peak, and a flawed extraction window can artificially inflate or deflate the peak areas of the key afucosylated and high mannose glycopairs reducing the accuracy of the analysis. Third, structural isomers and glycopairs with partially reduced disulfide bonds (Rd, +2 Da) lead to multiple chromatographic peaks for species categorized as the same glycopair. Finally, key afucosylated and high mannose glycopairs (shown in bold in FIG. 6B) have extremely low abundances, and often represent less than 5% of the population. Taken together, these challenges complicate the data analysis and quantitation and require the careful consideration of a highly-skilled analyst, and ultimately increase the time required for these overall method.

A new method was designed to include the preparation, separation, measurement, and analysis of a simplified sample comprising the components essential for quantitation of afucosylated and high mannose glycans (paired or unpaired) without an abundance of non-essential components. For example, in the previous method, every type of glycopair including the high mannose, and all fucosylated and afucosylated glycopairs were identified and quantified, and this degree of resolution was unnecessary for the purposes of determining levels of unpaired and paired afucosylated and high mannose glycopairs. Further, the separation prior to the MS-based detection was designed so the glycosylated Fc fragments eluted under one chromatographic peak in the total ion chromatogram which was chromatographically separated from the Fab region. In the previous HILIC-MS method, poor chromatographic resolution of key afucosylated and high mannose glycopairs from the abundant fucosylated glycopairs lead to difficulties determining the correct mass spectral extraction windows for accurate quantitation. To simplify analysis in the new method all glycosylated Fc fragments intentionally co-elute under one chromatographic peak in the total ion chromatogram. The mass spectra are extracted from the entire chromatographic peak of the Fc fragment and deconvoluted. The deconvoluted mass spectral peaks in the one deconvoluted mass spectrum are leveraged for identification and quantitation of the glycopairs. Using this approach, the Fc fragments are separated by their differences (delta) in molecular weights (MW). By intentionally removing the chromatographic separation of the glycosylated Fc fragments the data analysis is simplified, and subsequently quantitative accuracy and sample-to-sample comparability is improved. Additionally, the sample through-put is dramatically increased. An automated identification system for faster identification and quantitation of the paired and unpaired afucosylated and high mannose glycopairs was additionally included in the new method.

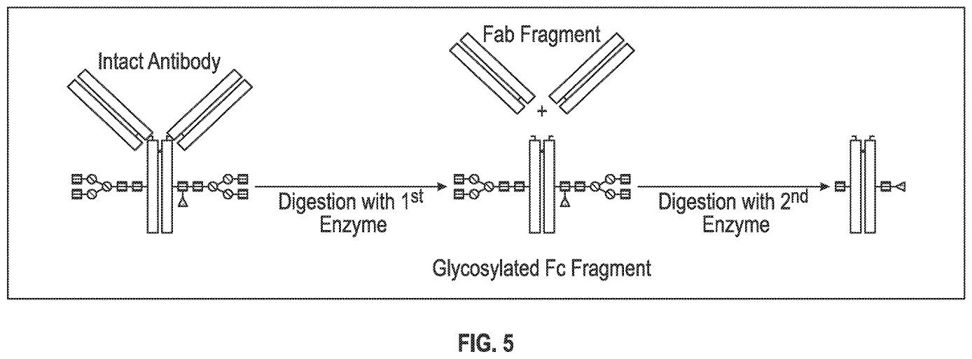

The new method comprised a two-enzyme digestion of the antibody composition before chromatographic separation and a MS-based detection. This two-enzyme digestion is depicted in FIG. 5. Briefly, samples of the antibody composition were treated with the enzyme IgdE (FabALACTICA® or FL from Genovis Inc., Cambridge, MA) which cleaves the heavy chain of IgG1 antibodies above the hinge to create Fab and Fc antibody fragments. The FL enzyme cleaves between the second and third amino acid of the IgG1 heavy chain sequence KTHTCPP (SEQ ID NO: 1). The digestion was carried out overnight at 37° C. A second digestion with EndoS (IgGZERO® from Genovis inc.), an IgG specific endoglycosidase S enzyme, was carried out for 30 min at 37° C. EndoS is an IgG-specific endoglycosidase acting on complex type N-glycans at the Fc-glycosylation site of IgGs. The enzyme hydrolyzes the β1,4 linkage between the core GlcNAc residues in the N-glycans, leaving the innermost GlcNAc intact on the Fc with or without a fucose. EndoS has limited activity on high mannose and hybrid glycans, this is leveraged to identify and quantify the high mannose glycopairs and subsequently the % relative abundance of paired and unpaired high mannose.

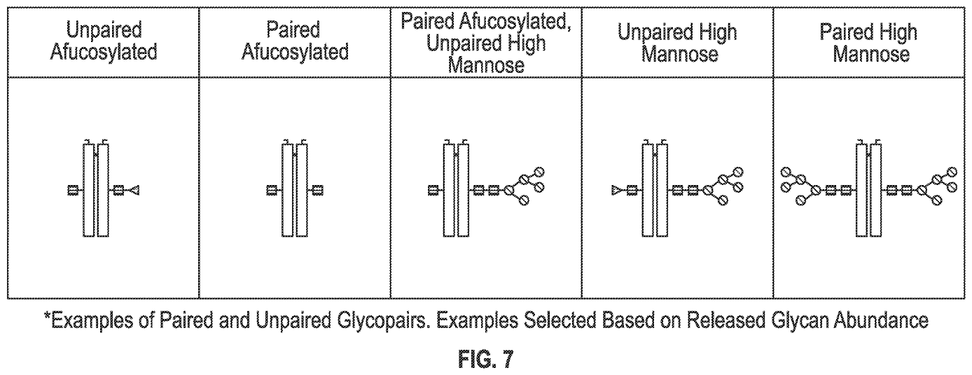

This two-enzyme digestion yielded a simplified population of glycopairs which could be categorized into one of two afucosylated glycopair classes: (1) paired afucosylated (both heavy chains of the Fc fragment were afucosylated) and (2) unpaired afucosylated (only one heavy chain of the Fc fragment was afucosylated, while the other heavy chain was fucosylated) (FIG. 7). Based on the assumption that both afucosylated and high mannose glycans are highly potent, pairs containing a combination of high mannose and afucosylated glycans were classed as afucosylated paired, unpaired high mannose, as appropriate and according to Table A.

EndoS has limited activity on high mannose and hybrid glycans, this was leveraged to identify high mannose glycopairs and quantify the high mannose glycan pairing. The population of individual Fc high mannose glycopairs could be categorized into one of two high mannose classes: (1) paired high mannose (both heavy chains of the Fc fragment contained high mannose glycans) and (2) unpaired high mannose (only one heavy chain of the Fc fragment contained high mannose, while the other heavy chain was fucosylated or afucosylated) (FIG. 7). A summary designation was given according to Table A and following the three rules described herein.

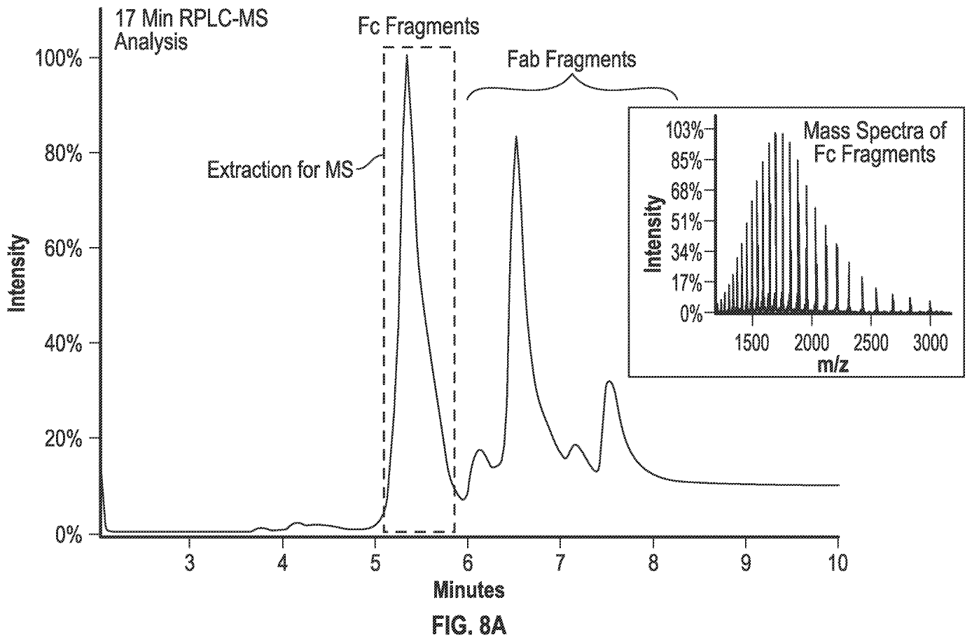

Following the two-enzyme digestion, resulting Fc and Fab fragments were chromatographically separated by using a rapid reversed phase liquid chromatography (RP-LC) separation with MS detection. This separation yielded one chromatographic peak comprising the Fc fragments that was well separated from the Fab fragments and treatment enzymes. A diagram of the chromatographic separation and MS analysis steps are shown in FIGS. 8A-8B.

The equipment used to perform steps of the method has the following capabilities:

Equipment capabilities:

UPLC system with column heating capabilities
MS system with an ESI source and high-resolution mass
analyzer (time-of-flight or orbitrap)
Temperature controlled autosampler
Reversed phase column

The autosampler temperature was held at 4° C. and an aliquot was injected and the separation was carried out using a 2.11 D×50 mm RP analytical column at 60° C. Mobile Phase A was 0.1% (v/v) trifluoroacetic: acid in water while Mobile Phase B was 0.1% (v/v) trifluoroacetic acid in acetonitrile. The mobile phase gradient example is provided below:

| | Flow | Mobile | Mobile |
|---|---|---|---|
| Time | Rate | Phase A | Phase B |
| (minutes) | (mL/minute) | (%) | (%) |
| 0.0 | 0.25 | 70 | 30 |
| 10 | 0.25 | 55 | 45 |
| 10.3 | 0.25 | 10 | 90 |
| 11.3 | 0.25 | 10 | 90 |
| 12 | 0.25 | 70 | 30 |
| 15 | 0.25 | 70 | 30 |
| 17 | 0.25 | 70 | 30 |

MS acquisition was performed using an Agilent 6545 XT Q-TOF with an AJS ESI source in positive ion mode. The source conditions were: gas temperature—325° C., drying and sheath gas flow—12 L/min, sheath gas temperature—300° C., nebulizer—40 psi, capillary voltage—5000 V, fragmentor voltage—280 V, and skimmer—120 V. Spectra were acquired with a m/z range of 1200-3200.

The chromatographic peak that contained the Fc fragments, was then selected for data analysis, the mass spectra of the one chromatographic peak that contained the Fc fragments was extracted and deconvoluted (FIGS. 8A-8B). The deconvolution was performed using Byos (Protein Metrics Inc, Cupertino, CA) the input m/z range was 1300-3200 m/z while the output range was 30-70 kDa. The intensity of each deconvoluted mass spectral peak of the antibody composition was employed for quantitation.

The EndoS treated glycopairs were identified by matching the molecular weights (MW) of the deconvoluted mass spectral peaks of the antibody composition against a custom-built database of theoretical MWs (Table 1). The glycan MWs used in this approach is the glycan MW associated with the N-linkage (native glycan MW—H₂O). The database comprised of the theoretical MWs of two EndoS treated glycans (glycan pairs) whereby complex glycans were hydrolyzed to either GlcNac or GlucNac+Fuc while high mannose and hybrid glycans remained intact. The database comprised of the theoretical MWs of approximately 40 individual EndoS glycan pairs classed as (1) afucosylated paired and (2) afucosylated unpaired and (3) high mannose paired and (4) high mannose unpaired (Table 1). Glycan pairs that have been hydrolyzed by EndoS are italicized in the subsequent text.

To facilitate the automated identification of the EndoS treated glycopairs (defined as the intact Fc and two glycan moieties) the amino acid sequence of the FL treated Fc was first loaded into the protein analysis software to generate a theoretical MW of the Fc, additionally the software was instructed to consider that all possible disulfide bonds remain intact and both C-terminal lysines were clipped. Second, the database of the theoretical MWs of EndoS glycan pairs was loaded into the protein analysis software as a list of custom modifications. Following this user input, the protein analysis software then automatically determined a list of theoretical MWs for the EndoS glycopairs (seen in Table 1). The software automatically identified the deconvoluted mass spectral peaks if the MW matched with a theoretical MW in the Custom-built database of EndoS glycopairs.

TABLE 1

| | EndoS | EndoS |
|---|---|---|
| | Glycan | Glycopair |
| | Pair MW (Da) | MW (Da) |
| Dominant Paired Fucosylated EndoS Glycan Pair | | |
| GlcNac/GlcNac + Fuc | 698.6 | 50901.2 |
| Paired Afucosylated EndoS Glycan Pairs | | |
| GlcNac/GlcNac | 406.4 | 50608.9 |
| M5/GlcNac | 1420.3 | 51622.8 |
| M6/GlcNac | 1582.4 | 51785 |
| M7/GlcNac | 1744.6 | 51947.1 |
| M8/GlcNac | 1906.7 | 52109.2 |
| A1G0M5/GlcNac | 1623.5 | 51826 |
| A1G0M5/A1G0M5 | 2840.6 | 53043.1 |
| A1G1M5/GlcNac | 1785.6 | 51988.2 |
| A1G1M5/A1G1M5 | 3164.8 | 53367.4 |
| M5/A1G0M5 | 2637.4 | 52839.9 |
| M6/A1G0M5 or M5/A1G1M5 | 2799.5 | 53002 |
| M7/A1G0M5 or M6/A1G1M5 | 2961.6 | 53164.2 |
| M8/A1G0M5 or M7/A1G1M5 | 3123.8 | 53326.3 |
| M8/A1G1M5 | 3285.9 | 53488.5 |
| Unpaired Afucosylated EndoS Glycan Pairs | | |
| GlcNac/GlcNac + Fuc | 552.5 | 50755.1 |
| GlcNac | 203.2 | 50405.7 |
| A1G0M5/GlcNac or GlcNac/A1G0M5F | 1769.6 | 51972.2 |
| A1G1M5/GlcNac + Fuc or GlcNac/A1G1M5F | 1931.8 | 52134.3 |
| GlcNac/A1G0M4F | 1753.6 | 51956.2 |
| GlcNac/A1S1M4F | 2060.9 | 52263.4 |
| GlcNac/A1G1S1M5F | 2223 | 52425.5 |
| Paired High Mannose EndoS Glycan Pairs | | |
| M5/M5 | 2434.2 | 52636.7 |
| M5/M6 | 2596.3 | 52798.9 |
| M5/M7 or FC, M6/M6 | 2758.5 | 52961 |
| M5/M8 or FC, M6/M7 | 2920.6 | 53123.1 |
| M6/M8 or FC, M7/M7 | 3082.7 | 53285.3 |
| M7/M8 | 3244.9 | 53447.4 |
| M8/M8 | 3407 | 53609.6 |
| Unpaired High Mannose EndoS Glycan Pairs | | |
| M5/GlcNac + Fuc | 1566.4 | 51769 |
| M6/GlcNac + Fuc | 1728.6 | 51931.1 |
| M7/GlcNac + Fuc | 1890.7 | 52093.2 |
| M8/GlcNac + Fuc | 2052.8 | 52255.4 |
| M5/A1G0M4F | 2621.4 | 52823.9 |
| M6/A1G0M4F or FC, M5/A1G0M5F | 2783.5 | 52986.1 |
| M7/A1G0M4F or FC, M6/A1G0M5F or FC + M5/A1G1M5F | 2945.7 | 53148.2 |
| M8/A1G0M4F or FC, M7/A1G0M5F or FC + M6/A1G1M5F | 3107.8 | 53310.3 |
| M8/A1G0M5F or FC + M7/A1G1M5F | 3269.9 | 53472.5 |
| M8/A1G1M5F | 3432.1 | 53634.6 |
| M5/A1S1M4F | 3074.8 | 53277.3 |
| M6/A1S1M4F or FC + M5/A1G1S1M5F | 3236.9 | 53439.4 |
| M7/A1S1M4F or FC + M6/A1G1S1M5F | 3399 | 53601.6 |
| M8/A1S1M4F or FC + M7/A1G1S1M5F | 3561.2 | 53763.7 |
| M8/A1G1S1M5F | 3723.3 | 53925.9 |

Note:
Additional MWs for EndoS treated glycopairs which have only a single glycoslyation or a bisecting GlcNac are included in the database but not listed above.

The most abundant EndoS glycopair is Fc, GlucNac+Fucose/GlucNac+Fucose which contains a fucosylated glycan on each heavy chain and is therefore not applicable to the determination of afucosylated or high mannose glycan pairing. In the previous study using HILIC-MS, the highly abundant and not applicable paired fucosylated glycopairs elute across the entire region of the chromatogram where the glycosylated Fc elutes often masking the presence of the species of interest (FIG. 6B). In, the current study, the EndoS treatment simplifies the multitude of highly abundant paired fucosylated glycopairs into a single species, Fc, GlucNac+Fucose/GlucNac+Fucose. This single deconvoluted mass peak is well separated, by the delta molecular weight, from the species of interest which facilitates streamlined data interpretation (FIG. 8B).

The abundance of the individual EndoS glycopairs was determined from the relative intensities or areas of the identified deconvoluted mass spectral peaks as follows:

$$\% Glycopair Intensity = Deconvoluted Peak Intensity / Total Deconvoluted Peak Intensity \times 100 \qquad [ Equation 1 ]$$

The abundence of paired afucosyated glycopairs was determined by summing the individual glycopairs categorized as paired afucosylated glycopairs, similarly the abundence of unpaired afucosyated glycopairs was determined by summing the individual glycopairs categorized as unpaired afucosylated glycopairs. To calculate the % relative abundence of the Unpaired Afucosylated Glycan Content of the antibody composition Equation 2 was employed:

$$\% Unpaired Afucosylated Glycan Content = ( \% Unpaired Afucosylated Glycopairs / \% Total Afucosylated Glycopairs ( Unpaired + Paired ) ) \times 100 \qquad [ Equation 2 ]$$

The abundence of paired high mannose glycopairs was determined by summing the individual glycopairs categorized as paired high mannose glycopairs, similarly the abundence of unpaired high mannose glycopairs was determined by summing the individual glycopairs categorized as unpaired high mannose glycopairs. To calculate the % relative abundence of the Unpaired High Mannose Glycan content of the antibody composition Equation 3 was employed:

$$\% Unpaired High Mannose Glycan Content = ( \% Unpaired High Mannose Glycopairs / \% Total High Mannose Glycopairs ( Unpaired + Paired ) ) \times 100 \qquad [ Equation 3 ]$$

In the previous study, a 95 min HILIC-MS analysis was employed to separate a highly complex population of glycopairs for the determination of the levels of afucoslyated and high mannose glycan pairing. This approach remains challenging as the highly abundant and not applicable paired fucosylated glycopairs elute across the entire glycoslyated Fc chromatographic region often masking the presence of the glycopairs of interest. Key high mannose and afucosylated glycopairs co-elute with and/or elute as sholders of the highly abundant paired fucosylated glycopairs making accurate quantitation challenging. This example demonstrates the feasibility of a streamlined method where the glycopair population is simplified prior to analysis with EndoS. A rapid 17-min chromatographic separation prior to MS-detection simplifies analysis by yielding one, well resolved, chromatographic peak of Fc fragments for further quantitation. The quantitation of the EndoS glycopairs is achieved using the deconvoluted mass spectral peaks where the highly abundent paired fucosylated species is a single deconvoluted mass spectral peak well separated, by MW, from the key afucosylated and high mannose glycopairs. The custom built EndoS glycan pair MW database facilities automated identification and quantation of the critical afucosylated and high mannose glycopairs. This new method affords a rapid analysis and increased quantitative accuracy when determining % relative abundance of the unpaired and paired afucosylated glycan content and % relative abundance of the unpaired and paired high mannose glycan content of an antibody composition.

### Example 2

This example describes an exemplary application of the simplified method of determining the % relative paired and unpaired afucosylated and high mannose glycan content for an antibody composition.

The new method described in Example 1 was carried out on a panel of antibody composition samples, wherein individual samples of the panel comprised an IgG1 monoclonal antibody produced by a particular clone or a sample of a reference product (RP) lot. Deconvoluted mass spectra for selected samples are shown in FIGS. 9A-9C. Data for paired afucosylated glycopairs and unpaired afucosylated glycopairs are provided in Tables 2A and 2B, respectively. Data for paired high mannose glycopairs and unpaired high mannose glycopairs are provided in Tables 2C and 21), respectively.

TABLE 2A

| | % FC, | % FC, | % Paired |
|---|---|---|---|
| | GlcNac/ | M5/ | Afucosylated |
| Sample | GlcNac | GlcNac | Glycopairs |
| Clone 113 | 0.59 | 0.36 | 0.95 |
| Clone 116 | 1.37 | 0.08 | 1.45 |
| Clone 126 | 0.73 | 0.42 | 1.15 |
| Clone 203 | 1.3 | 0.57 | 1.87 |
| Clone 236 | 0.17 | 0.36 | 0.53 |
| Clone 85 | 1.43 | 0.57 | 2.00 |
| Clone 93 | 1.65 | 0.76 | 2.41 |
| Clone 98 | 1.72 | 0.77 | 2.49 |
| Clone 160 | 0.71 | ND | 0.71 |
| Clone 160.2 | 0.07 | ND | 0.07 |
| Clone 182 | 0.72 | ND | 0.72 |
| Clone 260 | 1.47 | ND | 1.47 |
| RP15 | 1.21 | ND | 1.21 |
| RP16 | 1.23 | ND | 1.23 |
| RP44 | 1.07 | ND | 1.07 |
| RP51 | 0.94 | ND | 0.94 |
| RP54 | 1.34 | ND | 1.34 |

*Note:
Only the paired afucosylated glycopairs detected in 1 or more samples are listed. ND = Not detected for sample. Italicized glycans have been hydrolyzed by EndoS

TABLE 2B

| | % Fc, | % Fc, | % Fc, | |
|---|---|---|---|---|
| | GlcNac/ | A1G1M4/ | A1G1M5/ | % Paired |
| | GlcNac + | GlcNac + | GlcNac + | Afucosylated |
| Samples | Fuc | Fuc | Fuc | Glycopairs |
| Clone 113 | 9.75 | ND | ND | 9.75 |
| Clone 116 | 13.28 | ND | ND | 13.28 |
| Clone 126 | 10.26 | ND | ND | 10.26 |
| Clone 203 | 12.51 | ND | ND | 12.51 |
| Clone 236 | 8.34 | ND | ND | 8.34 |
| Clone 85 | 13.72 | ND | ND | 13.72 |
| Clone 93 | 14.58 | ND | ND | 14.58 |
| Clone 98 | 14.71 | ND | ND | 14.71 |
| Clone 160 | 6.19 | 0.4 | ND | 6.59 |
| Clone 160.2 | 3.78 | 0.74 | ND | 4.52 |
| Clone 182 | 5.09 | 0.05 | ND | 5.14 |
| Clone 260 | 9.9 | 1.86 | 0.23 | 11.99 |
| RP15 | 7.67 | ND | ND | 7.67 |
| RP16 | 8.11 | ND | ND | 8.11 |
| RP44 | 7.08 | ND | ND | 7.08 |
| RP51 | 7.15 | ND | ND | 7.15 |
| RP54 | 7.86 | ND | ND | 7.86 |

*Note:
Only the paired afucosylated glycopairs detected in 1 or more samples are listed. ND = Not detected for sample. Italicized glycans have been hydrolyzed by EndoS

TABLE 2C

| | | | | | | % Paired |
|---|---|---|---|---|---|---|
| | | | % Fc, | % Fc, | | High |
| | % Fc, | % Fc, | M6/M6 or | M5/M8 or | % Fc, | Mannose |
| Sample Name | M5/M5 | M5/M6 | Fc, M5/M7 | Fc, M6/M7 | M8/M8 | Glycopairs |
| Clone 113 | 0.02 | ND | 0.08 | ND | ND | 0.10 |
| Clone 116 | 0.13 | ND | 0.02 | ND | ND | 0.15 |
| Clone 126 | ND | ND | 0.02 | 0.02 | ND | 0.04 |
| Clone 203 | 0.2 | 0.01 | 0.01 | ND | ND | 0.22 |
| Clone 236 | ND | ND | 0.02 | ND | ND | 0.02 |
| Clone 85 | 0.21 | 0.01 | 0.01 | ND | ND | 0.23 |
| Clone 93 | 0.35 | ND | 0.02 | ND | ND | 0.37 |
| Clone 98 | 0.37 | 0.01 | ND | ND | ND | 0.38 |
| Clone 160 | ND | ND | 0.08 | ND | ND | 0.08 |
| Clone 160.2 | ND | ND | 0.07 | ND | 0.02 | 0.09 |
| Clone 182 | ND | ND | 0.12 | ND | 0.01 | 0.13 |
| Clone 260 | ND | ND | 0.04 | ND | ND | 0.04 |
| RP15 | 0.38 | ND | 0 | ND | ND | 0.38 |
| RP16 | 0.58 | ND | ND | ND | ND | 0.58 |
| RP44 | 0.19 | ND | ND | ND | ND | 0.19 |
| RP51 | 0.35 | ND | ND | ND | ND | 0.35 |
| RP54 | 0.24 | ND | 0.01 | ND | ND | 0.25 |

*Note:
Only the paired afucosylated glycopairs detected in 1 or more samples are listed. ND = Not detected for sample. Italicized glycans have been hydrolyzed by EndoS

TABLE 2D

| | | | | | % |
|---|---|---|---|---|---|
| | % Fc, | % Fc, | % Fc, | % Fc, | Unpaired |
| | M5/ | M6/ | M7/ | M8/ | High |
| | GlcNac + | GlcNac + | GlcNac + | GlcNac + | Mannose |
| Sample | Fuc | Fuc | Fuc | Fuc | Glycopairs |
| Clone 113 | 0.35 | ND | ND | 0.22 | 0.57 |
| Clone 116 | 0.67 | ND | ND | 0.07 | 0.74 |
| Clone 126 | ND | ND | ND | 0.2 | 0.20 |
| Clone 203 | 0.62 | ND | ND | 0.15 | 0.77 |
| Clone 236 | ND | ND | ND | 0.42 | 0.42 |
| Clone 85 | 0.41 | ND | ND | 0.15 | 0.56 |
| Clone 93 | 1.04 | ND | ND | 0.04 | 1.08 |
| Clone 98 | 1.04 | ND | ND | ND | 1.04 |
| Clone 160 | ND | 0.07 | ND | ND | 0.07 |
| Clone 160.2 | ND | 0.6 | ND | 0.36 | 0.96 |
| Clone 182 | 0.24 | 0.01 | ND | 0.16 | 0.41 |
| Clone 260 | ND | 1.1 | 0.45 | ND | 1.55 |
| RP15 | 1.09 | ND | ND | ND | 1.09 |
| RP16 | 1.22 | ND | ND | ND | 1.22 |
| RP44 | 0.82 | ND | ND | ND | 0.82 |
| RP51 | 0.96 | ND | ND | ND | 0.96 |
| RP54 | 0.92 | ND | ND | 0.02 | 0.94 |

*Note:
Only the paired afucosylated glycopairs detected in 1 or more samples are listed. ND = Not detected for sample. Italicized glycans have been hydrolyzed by EndoS

The relative unpaired and paired afucosylated glycan content and the relative unpaired and paired high mannose glycan content were calculated as described in Example 1 and the data is provided below in Table 3.

TABLE 3

| | | | % Unpaired | % Paired |
|---|---|---|---|---|
| | % Unpaired | % Paired | High | High |
| | Afucosylated | Afucosylated | Mannose | Mannose |
| | Glycan | Glycan | Glycan | Glycan |
| Sample | Content | Content | Content | Content |
| Clone 113 | 91.1 | 8.9 | 85.1 | 14.9 |
| Clone 116 | 90.2 | 9.8 | 83.1 | 16.9 |
| Clone 126 | 89.9 | 10.1 | 83.3 | 16.7 |
| Clone 203 | 87.0 | 13.0 | 77.8 | 22.2 |
| Clone 236 | 94.0 | 6.0 | 95.5 | 4.5 |
| Clone 85 | 87.3 | 12.7 | 70.9 | 29.1 |
| Clone 93 | 85.8 | 14.2 | 74.5 | 25.5 |
| Clone 98 | 85.5 | 14.5 | 73.2 | 26.8 |
| Clone 160 | 90.3 | 9.7 | 46.7 | 53.3 |
| Clone 160.2 | 98.4 | 1.6 | 92 | 8.0 |
| Clone 182 | 87.7 | 12.3 | 75.9 | 24.1 |
| Clone 260 | 89.1 | 10.9 | 97.5 | 2.5 |
| RP 15 | 86.4 | 13.6 | 74.1 | 25.9 |
| RP 16 | 86.8 | 13.2 | 67.8 | 32.2 |
| RP 44 | 86.9 | 13.1 | 81.2 | 18.8 |
| RP 51 | 88.4 | 11.6 | 73.3 | 26.7 |
| RP 54 | 85.4 | 14.6 | 79.0 | 21.0 |

Based on these data, the RP range of unpaired afucosylated glycan content was 85.4-88.4 and the range of unpaired high mannose glycan content was 67.8-81.2. The clones having the best unpaired glycan profiles are Clones 203, 116, 113, 126 and 236, with clones 116 and 126 having an unpaired afucosylated glycan content within the RP range and clone 203 having both unpaired afucosylated glycan content and unpaired high mannose glycan within the RP range.

### Example 3

This example describes an exemplary method for measuring ADCC activity levels.

ADCC activity levels (expressed as a % relative value) for a panel of samples of an IgG1 monoclonal antibody composition are determined using a quantitative cell-based assay that measures the ability of the antibody to mediate cell cytotoxicity in a dose-dependent manner of target cells stably expressing antigens while engaging FcγRIIIA (158V) receptors on NK92-M1 effector cells via the antibody Fc domain. These events lead to the activation of the effector cells and destruction of the target cells via exocytosis of the cytolytic granule complex perforin/granzyme. A schematic of the ADCC assay is provided in FIG. 10. Briefly, target cells are labeled with calcein-acetoxymethyl (calcein-AM), which readily enters the cells and is subsequently cleaved by intercellular esterases and trapped within the cells. When target cells are lysed, fluorescent calcein is released into the medium. The level of calcein released from lysed target cells is determined by measuring the fluorescence of the reaction supernatant in an Envision (Perkin Elmer) fluorescence plate reader. Each assay is performed in triplicate and the mean and standard deviation are recorded. The data are fitted to the mean fluorescence values using a constrained 4 parameter fit using SoftMaxPro software and reported as percentage ADCC activity relative to a reference standard as calculated by the ECSO standard/ECSO sample ratio.

### Example 4

HILIC is a quantitative analysis of the N-linked glycan distribution of the antibody and comprises three steps: (1) release and label N-linked glycans from reference and test samples using PNGase F and a fluorophore that can specifically derivatize free glycan, (2) load samples within the validated linear range onto a HILIC column, the labeled N-linked glycans are separated using a gradient of decreasing organic solvent, and (3) monitor elution of glycan species with fluorescence detector.

The standard and test samples are prepared by carrying out the following steps: (1) dilute samples and controls with water, (2) add PNGase F and incubate the samples and controls to release N-linked glycans, (3) mix with fluorophore labeling solution using a fluorophore such as 2-aminobenzoic acid. Vortex and incubate the samples and controls, (4) centrifuge down to pellet protein and remove supernatant, and (5) dry and reconstitute labeled glycans in the injection solution.

The solutions used in this assay are a Mobile Phase A (100 mM ammonium formate, target pH 3.0) and a Mobile Phase B (acetonitrile). The equipment used to perform steps of the method has the following capabilities:

Equipment capabilities:

UPLC system
Fluorescence detector set to appropriate excitation/emission
wavelength optimized to labeling fluorophore
Data collection system
Temperaturecontrolled autosampler
Hydrophilic interaction column

The instrument settings for HPLC using a hydrophilic interaction analytical BEH Glycan 1.7 μm column (2.1 mm ID×150 mm) and 2-aminobenzoic acid fluorophore labeling method are provided below:

| | | |
|---|---|---|
| Target sample load | 2 | μL |
| Column heater set point | 35° | C. |
| Auto-sampler set point | 10° | C. |
| Detection | Excitation 360 | nm |
| | Emission 425 | nm |

The mobile phase gradient example is provided below:

| | | Mobile | Mobile |
|---|---|---|---|
| Time | Flow Rate | Phase A | Phase B |
| (minutes) | (mL/minute) | (%) | (%) |
| 0.0 | 0.25 | 22.0 | 78.0 |
| 111.2 | 0.25 | 40.1 | 59.9 |
| 117.9 | 0.20 | 90.0 | 10.0 |
| 124.5 | 0.20 | 90.0 | 10.0 |
| 129.1 | 0.25 | 22.0 | 78.0 |
| 155 | 0.25 | 22.0 | 78.0 |

Reports of the results comprise the following format:

Report area % for high mannose glycans and afucosylated glycans*
% High mannose glycans = % M5 + % M6 + % M7 + % M8
% Afucosylated glycans = % A1G0 + % A2G0 + % A1G1a +
% A1G1b + % A2G1a + % A2G1b + % A1G1M4 + % A2G2 +
% A1G1M5 + % A1G1S1M5
*Calculation formulas depend on presence of individual high mannose and afucosylated glycans

### Example 5

This example describes the relationships of ADCC activity to % released high mannose glycans, % released afucosylated glycans, % unpaired high mannose glycans and % unpaired afucosylated glycans for an antibody composition.

The ADCC activity level of a panel of antibody compositions was determined by a cell-based ADCC assay. The % released high mannose glycans and % released afucosylated glycans were determined by the method described in Example 4. The ADCC activity levels, % released high mannose glycans, and % released afucosylated glycans of each antibody composition of the panel are provided in Table 4.

TABLE 4

| | % Released | % Released | % Relative |
|---|---|---|---|
| | Afucosylated | High Mannose | ADCC |
| | Glycans | Glycans | Response |
| Clone 203 | 7.7 | 2.6 | 181 |
| Clone 116 | 7.5 | 2.5 | 181 |
| Clone 113 | 5.7 | 2.8 | 150 |
| Clone 126 | 5.4 | 2.7 | 128 |
| Clone 236 | 4.5 | 1.8 | 117 |
| Clone 85 | 8.8 | 2.2 | 195 |
| Clone 93 | 8.9 | 3 | 197 |
| Clone 98 | 9.7 | 3.6 | 201 |
| Clone 160 | 5.9 | 0.6 | 114 |
| Clone 160.2 | 2.9 | 1.4 | 61 |
| Clone 182 | 4.7 | 1.2 | 97 |
| Clone 260 | 7.9 | 0.8 | 177 |
| RP15 | 4.3 | 2.7 | 95 |
| RP16 | 4.6 | 2.8 | 102 |
| RP44 | 4.0 | 1.9 | 83 |
| RP51 | 3.8 | 2.1 | 85 |
| RP54 | 4.6 | 1.9 | 85 |

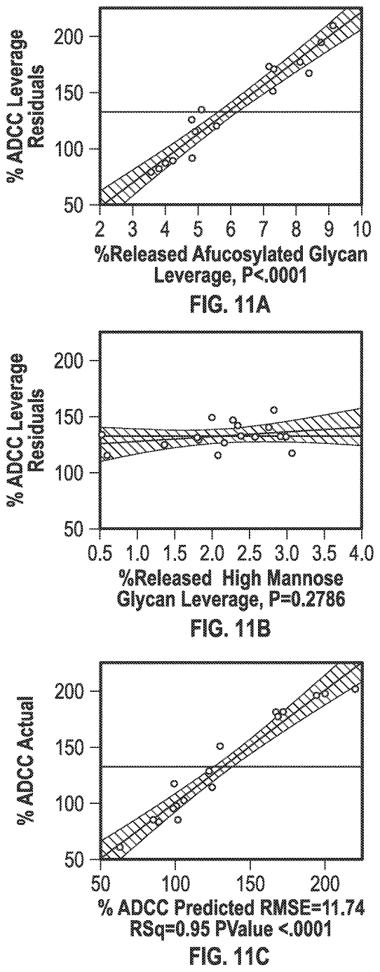

These data were analyzed using the JMP suite of computer programs for statistical analysis (SAS Institute, Cary, NC). FIG. 11A is an ADCC leverage plot for % released afucosylated glycans and FIG. 11B is a leverage plot for % released high mannose glycans. The best fit line is the solid diagonal line in the middle of the shaded area. As shown in FIGS. 11A and 11B, released afucosylated glycans demonstrated a statistically significant relationship with the ADCC activity level of the antibody composition (p<0.0001), whereas released high mannose glycans did not (p=0.2786).

The relationship between % ADCC and the % released afucosylated glycans and % released high mannose glycans may be described by Equation 4:

$$Predicted \% ADCC = - 5.8 + ( 21.7 * \% Released Afucosylated Glycans ) + ( 4.3 * \% Released High Mannose Glycans ) \qquad [ Equation 4 ]$$

Plugging the measured values for % released afucosylated glycans and % released high mannose glycans into Equation 4, a predicted % ADCC value was calculated for each sample. The actual % ADCC (as measured in the cell-based assay) was plotted against the predicted % ADCC (as calculated by Equation 4) and the plot is provided as FIG. 11C. Statistical parameters, including Root Mean Square Error (RMSE), r², and p-value, are shown in FIG. 11C. These results suggested that Equation 4 predicted the actual (measured) ADCC with accuracy and underlines the statistically significant correlation between % released afucosylated glycans and ADCC (p<0.0001). Higher levels of afucosylated glycans result in higher ADCC activity. The effect of high mannose on ADCC was weak and not statistically significant.

As the glycans are released from the Fc of the antibody composition in the approach outlined in Example 4 no glycan pairing information is captured and the leverage of both the unpaired and paired afucosylated glycans and the unpaired and paired high mannose glycans on ADCC activity cannot be determined. In the previous HILIC-MS method outlined in Example 1, an increased level of unpaired afucosylated glycans was shown to have an increased leverage on ADCC, likely because the percentage of afucosylated glycans is spread over a wider distribution of molecules of the antibody composition. Paired high mannose glycans has been shown to have a very low leverage on ADCC activity, however the structural reason for this is currently unknown.

The RP-LC MS approach outlined in Example 1 to measure the levels of paired and unpaired afucosylated and high mannose glycan content offers a powerful, but simplified approach to determine the relationship between ADCC activity and glycan pairing. The overall distribution of total afucosylated and total high mannose glycans determined by the approaches described in Example 1 and Example 4 should provide similar results. However, the RP-LC MS method was developed as a targeted workflow focused on the analysis of the unpaired afucosylated and high mannose glycan content, the full composition data are likely less accurate compared to the well-established released glycan assay described in Example 4. The optimal use of the glycan pairing data on the afucosylated and high mannose subgroups is in application in a form of a correction factor to the released afucosylated and high mannose glycan content determined using Example 4 to account for the contribution of glycan pairing. This correction can be used to determine the relationship between ADCC activity and unpaired afucosylated glycans and unpaired high mannose glycans for an antibody composition.

The paired and unpaired afucosylated glycan content and paired and unpaired high mannose glycan content of the same antibody composition were determined following the procedure outlined in Example 1. The results are provided in Table 3.

The fraction of paired afucosylated glycans was used to apply a correction to the released afucosylated glycan data in Table 4 to account for the portion of the antibody composition where the afucosylated glycans are paired (paired afucosylated fraction). Using Equation 5, the % Unpaired Afucosylated Glycans of the entire antibody composition can be determined.

$$\% Unpaired Afucosylated Glycans = \% released afucosylated glycans ( 1 - paired afucosylated fraction ) / ( 1 + paired afucosylated fraction ) \times 100 \qquad [ Equation 5 ]$$

The relative paired high mannose glycan content was used to apply a correction to the released high mannose glycan data in Table 4 to account for the portion of the antibody composition where the high mannose glycans are paired (paired high mannose fraction). Using Equation 6, the % Unpaired High Mannose Glycans of the entire antibody composition can be determined.

$$\% Unpaired High Mannose Glycans = \% released high mannose glycans ( 1 - paired high mannose fraction ) / ( 1 + paired high mannose fraction ) \times 100 \qquad [ Equation 6 ]$$

The ADCC activity levels, the % Unpaired Afucosylated Glycans and the % Unpaired High Mannose Glycans were calculated as described in Equation 5 and Equation 6 and the data is provided below in Table 5.

TABLE 5

| | % Unpaired | % Unpaired | % Relative |
|---|---|---|---|
| | Afucosylated | High Mannose | ADCC |
| Sample | Glycans | Glycans | Response |
| Clone 203 | 5.9 | 1.7 | 181 |
| Clone 116 | 6.2 | 1.8 | 181 |
| Clone 113 | 4.8 | 2.1 | 150 |
| Clone 126 | 4.4 | 1.9 | 128 |
| Clone 236 | 4 | 1.6 | 117 |
| Clone 85 | 6.8 | 1.2 | 195 |
| Clone 93 | 6.7 | 1.8 | 197 |
| Clone 98 | 7.2 | 2.1 | 201 |
| Clone 160 | 4.9 | 0.2 | 114 |
| Clone 160.2 | 2.8 | 1.2 | 61 |
| Clone 182 | 3.9 | 0.7 | 97 |
| Clone 260 | 6.8 | 0.8 | 177 |
| RP15 | 3.3 | 1.6 | 95 |
| RP16 | 3.5 | 1.4 | 102 |
| RP44 | 3.1 | 1.3 | 83 |
| RP51 | 3 | 1.2 | 85 |
| RP54 | 3.4 | 1.3 | 85 |

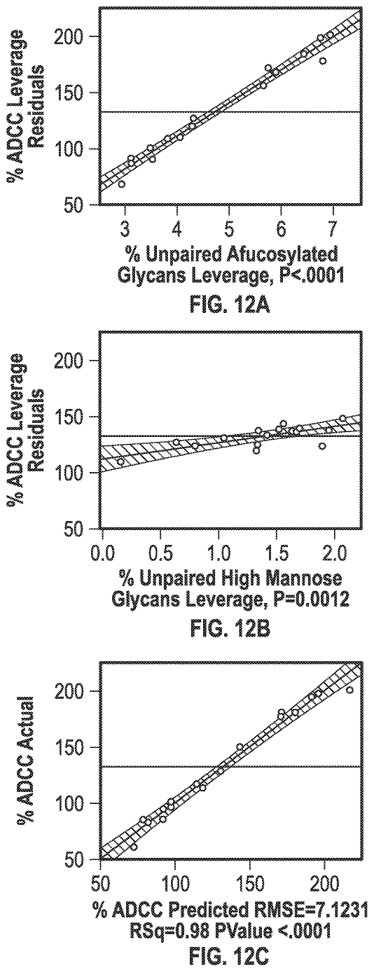

The data in Table 5 was then analyzed using the JMP suite of computer programs for statistical analysis. FIG. 12A is an ADCC leverage plot for % unpaired afucosylated glycans and FIG. 12B is a leverage plot for % unpaired high mannose glycans. The best fit line is the solid diagonal line in the middle of the shaded area. As shown in FIGS. 12A and 12B, both % unpaired afucosylated glycans and % unpaired high mannose glycans demonstrated a statistically significant relationship with ADCC activity levels (p<0.0001 for % unpaired afucosylated glycans and p=&0012 for % unpaired high mannose glycans). These results support that the % unpaired high mannose glycans do significantly contribute to ADCC activity.

Taken together, these data support that the pairing status matters. The overall % released high mannose glycan content did not appear to have a statistically significant relationship with the ADCC activity level, whereas the % unpaired high mannose glycan content was demonstrated to correlate with ADCC activity statistically significantly.

The relationship between % ADCC and the % unpaired afucosylated glycans and % unpaired high mannose glycans may be described by Equation 7:

$$Predicted \% ADCC = - 27.5 + ( 29.6 * \% Released Afucosylated Glycans ) + ( 14.5 * \% Released High Mannose Glycans ) \qquad [ Equation 7 ]$$

Plugging the measured values for % unpaired high mannose glycans and % unpaired afucosylated glycans into Equation 7, a predicted % ADCC value was calculated for each sample. The actual % ADCC (as measured in the cell-based assay) was plotted against the predicted % ADCC (as calculated by Equation 7) and the plot is provided as FIG. 12C. Statistical parameters, including Root Mean Square Error (RMSE), r², and p-value, are shown in FIG. 12C.

These results suggested that Equation 7 predicted the actual (measured) ADCC with accuracy and underlines the statistically significant direct correlation between % unpaired afucosylated glycans, % unpaired high mannose glycans, and ADCC activity level (p<0.0001). Higher levels of unpaired afucosylated glycan content and unpaired high mannoses content result in higher ADCC activity. The leverage of unpaired afucosylated glycans was stronger than the leverage of unpaired high mannose glycans on ADCC activity (29.6 and 14.5, respectively). Determining unpaired afucosylated glycans and unpaired high mannose glycans can predict ADCC levels of an antibody composition and thus predict product quality, e.g., quality of the antibody composition.

A similar approach can be used to correct the released afucosylated and high mannose glycan content to determine the paired afucosylated and paired high mannose glycan content respectively. For the current antibody composition, as the abundance of the paired glycan species are much lower, as seen in Table 3, a lower impact to ADCC activity is expected. Nonetheless, this approach can be employed to further understand the relationship between the paired glycan content and ADCC activity. It is expected that paired afucosylated glycans will have a statistically significant relationship with ADCC. As discussed above it is expected that the paired high mannose glycan content will have a very low leverage on ADCC activity.

These data support that glycan pairing analysis is important for understanding the structure-function relationship.

### Example 6

This example describes a method of determining the product quality during manufacture of an antibody composition.

Samples of a cell culture comprising Clone 203 producing an antibody are collected at different pre-harvest times post-inoculation and post-harvest prior to Protein A chromatography. The new method described in Example 1 is carried out on the obtained samples. Data for paired high mannose glycan content unpaired high mannose glycan pair content, paired afucosylated glycan content and unpaired afucosylated glycan pair content for each sample are recorded. The range of unpaired high mannose glycan content and unpaired afucosylated glycan content is pre-determined. The samples that are deemed to be outside this range are marked, and trouble-shooting on the corresponding manufacture days are monitored. It is discovered that a cell culture parameter shifts on this day and efforts to prevent the shift are taken. The process of collecting samples at different times pre-harvest and post-harvest as well as determining the unpaired high mannose glycan content and unpaired afucosylated glycan content of the samples are repeated. The samples are deemed as within the pre-determined range.

### Example 7

This example describes a method of determining the product quality of different manufacture lots of an antibody composition.

Samples of Lots 1-1.0 of an IgG1 monoclonal antibody are obtained following storage for 12, 24 and 36 months at 4° C. The new method described in Example 1 is carried out on the obtained samples. Data for paired high mannose glycan species, unpaired high mannose glycan pair content, paired afucosylated glycan content and unpaired afucosylated glycan pair content for each sample are recorded. Using Equation 7 and the data for % unpaired high mannose glycan molecules and % unpaired afucosylated glycan molecule, predicted ADCC activity levels for each lot are determined. Lots that are within the pre-determined ADCC range are determined as acceptable for administration to a patient, whereas lots that are outside the range are discarded.

### Example 8

This example describes a simplified method of determining paired glycan content and unpaired glycan content for a large scale-preparation of the IgG described in previous Examples 1-7,

Three batches of an IgG were made in large-scale bioreactors and samples from each batch were obtained and used in the two-enzyme digestion as essentially described in Example 1. Briefly, the samples were treated overnight with the enzyme IgdE (FabALACTICA® or FL from Genovis Inc., Cambridge, MA), and a second digestion was carried out with EndoS (IgGZERO® from Genovis Inc.,) for 30 min. Following the two-enzyme digestion, resulting Fc and Fab fragments were chromatographically separated by using a rapid reversed phase liquid chromatography (RP-LC) separation with MS detection, as essentially described in Example 1. This separation yielded one chromatographic peak comprising the Fc fragments that was well separated from the Fab fragments and treatment enzymes. The chromatographic peak that contained the Fc fragments, was then selected for data analysis, the mass spectra of the one chromatographic peak that contained the Fc fragments was extracted and deconvoluted, as essentially described in Example 1. The EndoS treated glycopairs were identified by matching the molecular weights (MW) of the deconvoluted mass spectral peaks of the antibody composition against a custom-built database of theoretical MWs (Table 1), as essentially described in Example 1. The data for unpaired afucosylated glycopairs, paired afucosylated glycopairs, unpaired high mannose glycopairs and paired afucosylated glycopairs are provided in Table 6.

TABLE 6

| | 500 L | 500 L | 500 L |
|---|---|---|---|
| Detected EndoS Glycopairs | Batch1 | Batch2 | Batch3 |
| FC, M5/M5 | 0.53 | 0.73 | 0.76 |
| % Paired High Mannose Glycopairs | 0.53 | 0.73 | 0.76 |
| FC, M5/GlcNac + Fuc | 1.35 | 1.35 | 0.97 |
| FC, M8/GlcNac + Fuc | 0.72 | 0.60 | 0.44 |
| % Unpaired High Mannose Glycopairs | 2.07 | 1.95 | 1.41 |
| FC, GlcNac/GlcNac + Fuc | 8.71 | 8.66 | 8.2 |
| % Unpaired Afucosylated Glycopairs | 8.71 | 8.66 | 8.20 |
| FC, GlcNac/GlcNac | 0.61 | 0.67 | 0.58 |
| FC, M5/GlcNac + Fuc | 0.46 | 0.43 | 0.19 |
| % Paired Afucosylated Glycopairs | 1.07 | 1.10 | 0.77 |

The relative unpaired and paired afucosylated glycan content and the relative unpaired and paired high mannose glycan content were calculated as described in Example 1 and the data is provided below in Table 7.

TABLE 7

| Relative Unpaired and | 500 L | 500 L | 500 L |
|---|---|---|---|
| Paired Glycan Content | Batch1 | Batch2 | Batch3 |
| % Unpaired Afucosylated | 89.1 | 88.7 | 91.4 |
| % Unpaired High Mannose | 79.6 | 72.8 | 65.0 |
| % Paired Afucosylated | 10.9 | 11.3 | 8.6 |
| % Paired High Mannose | 20.4 | 27.2 | 35.0 |

This example demonstrated that the new, simplified method of determining the relative unpaired glycan content can be carried out with a large-scale preparation of an IgG antibody composition.

### Example 9

This example describes a simplified method of determining paired glycan content and unpaired glycan content for an antibody composition comprising an IgG different from the IgG described in Examples 1-8. The IgG of this example targeted an antigen different from the target of the IgG of Examples 1-8.

Samples of an antibody composition comprising an IgG different from the IgG described in Examples 1-8 were used in a two-enzyme digestion as essentially described in Example 1. Briefly, the samples were treated overnight with the enzyme IgdE (FabALACTICA® or FL from Genovis Inc., Cambridge, MA), and a second digestion was carried out with EndoS (IgGZERO® from Genovis Inc.,) for 30 min. Following the two-enzyme digestion, resulting Fc and Fab fragments were chromatographically separated by using a rapid reversed phase liquid chromatography (RP-LC) separation with MS detection, as essentially described in Example 1. This separation yielded one chromatographic peak comprising the Fc fragments that was well separated from the Fab fragments and treatment enzymes. The chromatographic peak that contained the Fc fragments, was then selected for data analysis, the mass spectra of the one chromatographic peak that contained the Fc fragments was extracted and deconvoluted, as essentially described in Example 1. The EndoS treated glycopairs were identified by matching the molecular weights (MW) of the deconvoluted mass spectral peaks of the antibody composition against a custom-built database of theoretical MWs (Table 1), as essentially described in Example 1. The data for unpaired afucosylated glycopairs, paired afucosylated glycopairs, unpaired high mannose glycopairs and paired afucosylated glycopairs are provided in Table 8.

TABLE 8

| | Relative |
|---|---|
| Detected EndoS Glycopairs | Abundance (%) |
| FC, M5/M5 | 0.01 |
| % Paired High Mannose Glycopairs | 0.01 |
| FC, M5/GlcNac + Fuc | 0.53 |
| FC, M8/GlcNac + Fuc | 0.07 |
| % Unpaired High Mannose Glycopairs | 0.60 |
| FC, GlcNac/GlcNac + Fuc | 6.04 |
| % Unpaired Afucosylated Glycopairs | 6.04 |
| FC, GlcNac/GlcNac | 1.19 |
| FC, GlcNac | 0.22 |
| % Paired Afucosylated Glycopairs | 1.41 |

The relative unpaired and paired afucosylated glycan content and the relative unpaired and paired high mannose glycan content were calculated as described in Example 1 and the data is provided below in Table 9.

TABLE 9

| | Relative |
|---|---|
| Relative Unpaired and Paired Glycan Content | Abundance (%) |
| % Unpaired Afucosylated Glycan Content | 81.1 |
| % Unpaired High Mannose Glycan Content | 98.4 |
| % Paired Afucosylated Glycan Content | 18.9 |
| % Paired High Mannose Glycan Content | 1.6 |

This example demonstrated that the new, simplified method of determining the relative unpaired glycan content can be used with IgG antibody compositions other than the ones described in Examples 1-8.

### Example 10

This example describes a simplified method of determining paired glycan content and unpaired glycan content for an antibody composition comprising an IgG of the same isotype and antigen-specificity as the IgG of Examples 1-7 but different allotype (e.g., comprising a modified Fc region sequence, relative to the IgG of Examples 1-7).

Samples of an antibody composition comprising an IgG of a different allotype (relative to the IgG described in Examples 1-7) were used in a two-enzyme digestion as essentially described in Example 1. Briefly, the samples were treated overnight with the enzyme IgdE (FabALACTICA® or FL from Genovis Inc., Cambridge, MA), and a second digestion was carried out with EndoS (IgGZERO® from Genovis Inc.,) for 30 min. Following the two-enzyme digestion, resulting Fc and Fab fragments were chromatographically separated by using a rapid reversed phase liquid chromatography (RP-LC) separation with MS detection, as essentially described in Example 1. This separation yielded one chromatographic peak comprising the Fc fragments that was well separated from the Fab fragments and treatment enzymes. The chromatographic peak that contained the Fc fragments, was then selected for data analysis, the mass spectra of the one chromatographic peak that contained the Fc fragments was extracted and deconvoluted, as essentially described in Example 1. The EndoS treated glycopairs were identified by matching the molecular weights (MW) of the deconvoluted mass spectral peaks of the antibody composition against a custom-built database of theoretical MWs, as essentially described in Example 1, but the custom-built database used in this example is described in Table 10. The data for unpaired afucosylated glycopairs, paired afucosylated glycopairs, unpaired high mannose glycopairs and paired afucosylated glycopairs are provided in Table 11.

TABLE 10

| | EndoS | EndoS |
|---|---|---|
| | Glycan Pair | Glycopair |
| | MW (Da) | MW (Da) |
| Dominant Paired Fucosylated | | |
| EndoS Glycan Pair | | |
| GlcNac/GlcNac + Fuc | 698.6 | 50837.1 |
| Paired Afucosylated EndoS Glycan Pairs | | |
| GlcNac/GlcNac | 406.4 | 50544.8 |
| M5/GlcNac | 1420.3 | 51558.7 |
| M6/GlcNac | 1582.4 | 51720.8 |
| M7/GlcNac | 1744.6 | 51883.0 |
| M8/GlcNac | 1906.7 | 52045.1 |
| A1G0M5/GlcNac | 1623.5 | 51761.9 |
| A1G0M5/A1G0M5 | 2840.6 | 52979.0 |
| A1G1M5/GlcNac | 1785.6 | 52062.2 |
| A1G1M5/A1G1M5 | 3164.8 | 53579.6 |
| M5/A1G0M5 | 2637.4 | 52775.8 |
| M6/A1G0M5 or M5/A1G1M5 | 2799.5 | 52937.9 |
| M7/A1G0M5 or M6/A1G1M5 | 2961.6 | 53100.1 |
| M8/A1G0M5 or M7/A1G1M5 | 3123.8 | 53262.2 |
| M8/A1G1M5 | 3285.9 | 53562.5 |
| Unpaired Afucosylated EndoS Glycan Pairs | | |
| GlcNac/GlcNac + Fuc | 552.5 | 50487.7 |
| GlcNac | 203.2 | 50341.6 |
| A1G0M5/GlcNac or GlcNac/A1G0M5F | 1769.6 | 51908.0 |
| A1G1M5/GlcNac + Fuc or | 1931.8 | 52070.2 |
| GlcNac/A1G1M5F | | |
| GlcNac/A1G0M4F | 1753.6 | 51892.0 |
| GlcNac/A1S1M4F | 2060.9 | 52199.3 |
| GlcNac/A1G1S1M5F | 2223 | 52361.4 |
| Paired High Mannose EndoS Glycan Pairs | | |
| M5/M5 | 2434.2 | 52572.6 |
| M5/M6 | 2596.3 | 52734.7 |
| M5/M7 or FC, M6/M6 | 2758.5 | 52896.9 |
| M5/M8 or FC, M6/M7 | 2920.6 | 53059.0 |
| M6/M8 or FC, M7/M7 | 3082.7 | 53221.1 |
| M7/M8 | 3244.9 | 53383.3 |
| M8/M8 | 3407 | 53545.4 |
| Unpaired High Mannose EndoS Glycan Pairs | | |
| M5/GlcNac + Fuc | 1566.4 | 51704.8 |
| M6/GlcNac + Fuc | 1728.6 | 51867.0 |
| M7/GlcNac + Fuc | 1890.7 | 52029.1 |
| M8/GlcNac + Fuc | 2052.8 | 52191.3 |
| M5/A1G0M4F | 2621.4 | 52759.8 |
| M6/A1G0M4F or FC, M5/A1G0M5F | 2783.5 | 52921.9 |
| M7/A1G0M4F or FC, M6/A1G0M5F or | 2945.7 | 53084.1 |
| FC + M5/A1G1M5F | | |
| M8/A1G0M4F or FC, M7/A1G0M5F or | 3107.8 | 53246.2 |
| FC + M6/A1G1M5F | | |
| M8/A1G0M5F or FC + M7/A1G1M5F | 3269.9 | 53408.3 |
| M8/A1G1M5F | 3432.1 | 53570.5 |
| M5/A1S1M4F | 3074.8 | 53213.2 |
| M6/A1S1M4F or FC + M5/A1G1S1M5F | 3236.9 | 53375.3 |
| M7/A1S1M4F or FC + M6/A1G1S1M5F | 3399.0 | 53537.5 |
| M8/A1S1M4F or FC + M7/A1G1S1M5F | 3561.2 | 53699.6 |
| M8/A1G1S1M5F | 3723.3 | 53861.7 |

TABLE 11

| | Relative |
|---|---|
| Detected EndoS Glycopairs | Abundance (%) |
| FC, M5/M5 | 0.05 |
| FC, M6/M6 | 0.08 |
| % Paired High Mannose Glycopairs | 0.05 |
| FC, M5/GlcNac + Fuc | 0.66 |
| FC, M8/GlcNac + Fuc | 0.02 |
| % Unpaired High Mannose Glycopairs | 0.68 |
| FC, GlcNac/GlcNac + Fuc | 3.15 |
| % Unpaired Afucosylated Glycopairs | 3.15 |
| FC, GlcNac/GlcNac | 0.53 |
| FC, GlcNac | 0.08 |
| % Paired Afucosylated Glycopairs | 0.61 |

The relative unpaired and paired afucosylated glycan content and the relative unpaired and paired high mannose glycan content were calculated as described in Example 1 and the data is provided below in Table 12.

TABLE 12

| | Relative |
|---|---|
| Relative Unpaired and Paired Glycan Content | Abundance (%) |
| % Unpaired Afucosylated Glycan Content | 83.8 |
| % Unpaired High Mannose Glycan Content | 93.2 |
| % Paired Afucosylated Glycan Content | 16.2 |
| % Paired High Mannose Glycan Content | 6.8 |

### Example 11

This example describes a simplified method of determining paired glycan content and unpaired glycan content for a panel of antibody composition samples, wherein each individual sample of the panel comprised an IgG1 monoclonal antibody produced by a particular clone or was a sample of a reference product (RP) lot.

Samples of the panel were used in a two-enzyme digestion as essentially described in Example 1. Briefly, the samples were treated overnight with the enzyme IgdE (FabALACTICA® or FL from Genovis Inc., Cambridge, MA), and a second digestion was carried out with EndoS (IgGZERO® from Genovis Inc.,) for 30 min. Following the two-enzyme digestion, resulting Fc and Fab fragments were chromatographically separated by using a rapid reversed phase liquid chromatography (RP-LC) separation with MS detection, as essentially described in Example 1. This separation yielded one chromatographic peak comprising the Fc fragments that was well separated from the Fab fragments and treatment enzymes. The chromatographic peak that contained the Fc fragments, was then selected for data analysis, the mass spectra of the one chromatographic peak that contained the Fc fragments was extracted and deconvoluted, as essentially described in Example 1. The EndoS treated glycopairs were identified by matching the molecular weights (MW) of the deconvoluted mass spectral peaks of the antibody composition against a custom-built database of theoretical MWs (Table 10), as essentially described in Example 1. The data for unpaired afucosylated glycopairs, paired afucosylated glycopairs, unpaired high mannose glycopairs and paired afucosylated glycopairs are provided in Table 13.

TABLE 13

| | Lot 1 | Lot 1 | Lot 3 | Lot 4 | Lot 5 | Lot 6 |
|---|---|---|---|---|---|---|
| Detected EndoS Glycopairs | Relative Abundance (%) | | | | | |
| FC, M5/M5 | 0.07 | 0.14 | 0.03 | 0.09 | 0.1 | 0.12 |
| % Paired High Mannose Glycopairs | 0.07 | 0.14 | 0.03 | 0.09 | 0.10 | 0.12 |
| FC, M5/GlcNac + Fuc | 0.83 | 2.03 | ND | 1.47 | 1.53 | 1.95 |
| FC, M7/GlcNac + Fuc | ND | ND | ND | ND | 0.21 | ND |
| % Unpaired High Mannose Glycopairs | 0.83 | 2.03 | 0.00 | 1.47 | 1.74 | 1.95 |
| FC, GlcNac/GlcNac + Fuc | 7.17 | 1.71 | 7.16 | 4.07 | 4.4 | 2.03 |
| FC, A1G0M5/GlcNac + Fuc | ND | ND | ND | 0.27 | 0.52 | ND |
| FC, A1G1MS/GlcNac + Fuc | ND | ND | ND | 0.09 | ND | ND |
| % Unpaired Afucosylated Glycopairs | 7.17 | 1.71 | 7.16 | 4.43 | 4.92 | 2.03 |
| FC, GlcNac | 0.41 | ND | ND | 0.5 | 0.58 | ND |
| FC, GlcNac/GlcNac | 0.94 | 0.26 | 1 | 0.81 | 0.82 | 0.21 |
| FC, A1G1M5/A1G1M5 | ND | ND | 0.01 | ND | 0.02 | ND |
| FC, M5/GlcNac | ND | NO | ND | 0.35 | 0.41 | ND |
| FC, M8/GlcNac | ND | ND | ND | 0.18 | ND | ND |
| % Paired Afucosylated Glycopairs | 1.35 | 0.26 | 1.01 | 1.84 | 1.83 | 0.21 |
| Relative Unpaired and Paired Glycan Content | Lot 1 | Lot 1 | Lot 3 | Lot 4 | Lot 5 | Lot 6 |

The relative unpaired and paired afucosylated glycan content and the relative unpaired and paired high mannose glycan content were calculated as described in Example 1 and the data is provided below in Table 14.

TABLE 14

| Relative Unpaired and | Relative Abundance (%) | | | | | |
|---|---|---|---|---|---|---|
| Paired Glycan Content | Lot 1 | Lot 1 | Lot 3 | Lot 4 | Lot 5 | Lot 6 |
| % Unpaired Afucosylated Glycan Content | 84.2 | 86.8 | 87.6 | 70.7 | 72.9 | 90.6 |
| % Unpaired High Mannose Glycan Content | 92.2 | 93.5 | 0.0 | 94.2 | 94.6 | 94.2 |
| % Paired Afucosylated Glycan Content | 15.8 | 13.2 | 12.4 | 29.3 | 27.1 | 9.4 |
| % Paired High Mannose Glycan Content | 7.8 | 6.5 | 100.0 | 5.8 | 5.4 | 5.8 |

### Example 12

This example describes a simplified method of determining paired high mannose glycan content and unpaired high mannose glycan content for an antibody composition comprising a completely afucosylated IgG.

Samples of an antibody composition comprising completely afucosylated IgG were used in a two-enzyme digestion as essentially described in Example 1. Briefly, the samples were treated overnight with the enzyme IgdE (FabALACTICA® or FL from Genovis Inc., Cambridge, MA), and a second digestion was carried out with EndoS (IgGZERO® from Genovis Inc.,) for 30 min. Following the two-enzyme digestion, resulting Fc and Fab fragments were chromatographically separated by using a rapid reversed phase liquid chromatography (RP-LC) separation with MS detection, as essentially described in Example 1. This separation yielded one chromatographic peak comprising the Fc: fragments that was well separated from the Fab fragments and treatment enzymes. The chromatographic peak that contained the Fc fragments, was then selected for data analysis, the mass spectra of the one chromatographic peak that contained the Fc fragments was extracted and deconvoluted, as essentially described in Example 1. The EndoS treated glycopairs were identified by matching the molecular weights (MW) of the deconvoluted mass spectral peaks of the antibody composition against a custom-built database of theoretical MWs (Table 15), as essentially described in Example 1. The data for unpaired high mannose glycopairs and paired afucosylated glycopairs are provided in Table 16.

TABLE 15

| | EndoS | EndoS |
|---|---|---|
| | Glycan Pair | Glycopair |
| | MW (Da) | MW (Da) |
| Dominant Paired Afucosylated | | |
| EndoS Glycan Pair | | |
| GlcNac/GlcNac | 406.4 | 50544.8 |
| Paired Unpaired High Mannose | | |
| EndoS Glycan Pairs | | |
| M5/GlcNac | 1420.3 | 51558.7 |
| M6/GlcNac | 1582.4 | 51720.8 |
| M7/GlcNac | 1744.6 | 51883.0 |
| M8/GlcNac | 1906.7 | 52045.1 |
| M5/A1G0M5 | 2637.4 | 52775.8 |
| M6/A1G0M5 or M5/A1G1M5 | 2799.5 | 52937.9 |
| M7/A1G0M5 or M6/A1G1M5 | 2961.6 | 53100.0 |
| M8/A1G0M5 or M7/A1G1M5 | 3123.8 | 53262.2 |
| M8/A1G1M5 | 3285.9 | 53424.3 |
| Paired High Mannose EndoS Glycan Pairs | | |
| M5/M5 | 2434.2 | 52572.6 |
| M5/M6 | 2596.3 | 52734.7 |
| M5/M7 or FC, M6/M6 | 2758.5 | 52896.9 |
| M5/M8 or FC, M6/M7 | 2920.6 | 53059.0 |
| M6/M8 or FC, M7/M7 | 3082.7 | 52896.9 |
| M7/M8 | 3244.9 | 53059.0 |
| M8/M8 | 3407 | 53221.1 |

TABLE 16

| | % Relative |
|---|---|
| Detected EndoS Glycopairs | Abundance |
| FC, M5/M5 | 1.1 |
| % Paired High Mannose Glycopairs | 1.1 |
| FC, M5/GlcNac + Fuc | 3.9 |
| FC, M5/GlcNac + Fuc | 0.6 |
| FC, M8/GlcNac + Fuc | 0.2 |
| % Unpaired High Mannose Glycopairs | 4.6 |

The relative unpaired and paired high mannose glycan content were calculated as described in Example 1 and the data is provided below in Table 17.

TABLE 17

| Relative Unpaired and | % Relative |
|---|---|
| Paired Glycan Content | Abundance |
| % Unpaired High Mannose | 80.4 |
| % Paired High Mannose | 19.6 |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms including the indicated component(s) but not excluding other elements (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. A method of determining the relative unpaired glycan content of an IgG antibody composition, comprising
a. treating the IgG antibody composition with two enzymes to form a mixture of Fab fragments and Fc fragments, wherein one enzyme cleaves an antibody heavy chain at a site N-terminal to the hinge region disulfide linkages and the other enzyme cleaves the β1,4 linkage between core GlcNAc residues to form Fc fragments each comprising a pair of core glycan structures,
b. separating the Fab fragments from the Fc fragments, and
c. quantifying the abundance of
i. paired afucosylated Fc fragments,
ii. unpaired afucosylated Fc fragments,
iii. paired high mannose Fc fragments, and/or
iv. unpaired high mannose Fc fragments,
to determine the relative unpaired afucosylated (AF) glycan content and relative unpaired high mannose (HM) glycan content.

2. The method of claim 1, wherein one enzyme is a cysteine protease.

3. The method of claim 2, wherein the cysteine protease cleaves at a site between Thr and His or between Lys and Thr of the sequence KTHTCPP (SEQ ID NO: 1) of an IgG1 antibody heavy chain.

4. The method of claim 2 or 3, comprising treating the IgG antibody composition with the cysteine protease for at least 8 hours or at least 12 hours.

5. The method of any one of the preceding claims, wherein the other enzyme is an IgG-specific enzyme.

6. The method of claim 5, wherein the IgG-specific enzyme is an endoglycosidase.

7. The method of claim 6, wherein the endoglycosidase is an Endo S endoglycosidase.

8. The method of any one of claims 5-7, comprising treating the mixture with the IgG-specific enzyme for less than 1 hour.

9. The method of claim 8, comprising treating the IgG antibody composition with the IgG-specific enzyme for about 30 minutes.

10. The method of any one of the preceding claims, wherein the Fab fragments are separated from the Fc fragments by a chromatography.

11. The method of claim 10, wherein the chromatography is a reversed phase liquid chromatography.

12. The method of claim 10 or 11, comprising performing mass spectroscopy on a chromatographic fraction comprising the Fc fragments to obtain one or more mass spectral peaks.

13. The method of claim 12, comprising deconvoluting the mass spectral peaks to obtain deconvoluted mass spectral peaks.

14. The method of claim 13, comprising matching the molecular weight of each deconvoluted mass spectral peak against a database of glycan pairs and the associated molecular weight to identify the glycan pair.

15. The method of any one of the preceding claims, wherein (b) and (c) occurs in less than 2 hours.

16. The method of claim 15, wherein (b) and (c) occurs in less than 75 minutes.

17. The method of claim 16, wherein (b) and (c) occurs in less than 60 minutes.

18. A method of preparing an IgG antibody composition having an ADCC activity within a target range, comprising determining the relative unpaired glycan content of a sample of the IgG antibody composition in accordance with a method of any one of claims 1 to 17, and comparing the relative unpaired AF glycan content and/or relative unpaired HM glycan content of the IgG antibody composition to a target range of relative unpaired AF glycan content and/or relative unpaired HM glycan.

19. A method of analyzing an IgG antibody composition, comprising determining the relative unpaired glycan content of a sample of the IgG antibody composition in accordance with a method of any one of claims 1 to 17, and, optionally, comparing the relative unpaired AF glycan content and/or relative unpaired HM glycan content of the IgG antibody composition to the relative unpaired AF glycan content and/or relative unpaired HM glycan content of a reference product.

20. The method of claim 18 or 19, wherein the method is a part of a manufacturing process for the IgG antibody composition.

21. The method of any one of claims 18 to 20, wherein the method is performed in real time during manufacturing of the IgG antibody composition.

22. The method of any one of claims 18 to 21, wherein the sample is a sample of in-process material.

23. The method of any one of claims 18 to 22, wherein the relative unpaired glycan content is determined pre-harvest or post-harvest.

24. The method of claim 23, wherein the relative unpaired glycan content is determined post-harvest.

25. The method of any one of claims 13 to 25, wherein the sample is obtained from a manufacturing lot.

26. A method of monitoring production of an IgG antibody composition, comprising determining the relative unpaired glycan content of an IgG antibody composition in accordance with a method of any one of claim 1 to 17, for a first sample obtained at a first timepoint and for a second sample taken at a second timepoint which is different from the first timepoint, and, optionally, comparing the relative unpaired AF glycan content and/or relative unpaired HM glycan content of the first sample to the relative unpaired AF glycan content and/or relative unpaired HM glycan content of the second sample.

27. The method of claim 26, wherein each of the first sample and second sample is a sample of in-process material.

28. The method of claim 26, wherein the first sample is a sample of in-process material and the second sample is a sample of a manufacturing lot.

29. The method of claim 26, wherein the first sample is a sample obtained before one or more conditions of the cell culture are modified and the second sample is a sample obtained after the one or more conditions of the cell culture are modified.

30. A method of producing an IgG antibody composition, comprising (A) determining relative unpaired glycan content of a sample of the IgG antibody composition in accordance with a method of any one of claims 1-17, wherein the sample is a sample of in-process material, wherein, when the relative unpaired glycan content is determined as outside a target range, the method further comprises (B) modifying one or more conditions of the cell culture to obtain a modified cell culture and determining the relative unpaired glycan content, and optionally, repeating (A) and (B) until the relative unpaired glycan content is within the target range.

31. The method of claim 30, wherein one or more conditions of the cell culture are modified to primarily change the relative unpaired HM glycan content to achieve the target range of relative unpaired glycan content.

32. The method of claim 30, wherein one or more conditions of the cell culture are modified to primarily change the relative unpaired AF glycan content to achieve the target range of relative unpaired glycan content.
